(19) United States
(12) Patent Application Publication
(10) Pub. No.: US 2022/0409617 A1
(43) Pub. Date: Dec. 29, 2022

(54) Drug Formulations and Methods of Treatment for Metabolic Disorders

(71) Applicant: The Regents of the University of California, Oakland, CA (US)

(72) Inventor: Aimee Edinger, Irvine, CA (US)

(73) Assignee: The Regents of the University of California, Oakland, CA (US)

(21) Appl. No.: 17/756,013

(22) PCT Filed: Nov. 13, 2020
(86) PCT No.: PCT/US20/60513

§ 371 Date: May 13, 2022

Related U.S. Application Data
(60) Provisional application No. 62/934,941, filed on Nov. 13, 2019.

(51) Int. Cl. A61K 31/506; A61K 31/40; A61K 31/519; A61K 31/4196; A61K 31/5415; A61K 31/5377; A61K 31/175; A61P 3/10; A61P 3/04

(52) U.S. Cl. CPC A61K 31/506; A61K 31/40; A61K 31/519; A61K 31/4196; A61K 31/5415; A61K 31/5377; A61K 31/175; A61P 3/10; A61P 3/04

(57) ABSTRACT

Methods of treatment of metabolic disorders with various compounds are provided. Subjects having a metabolic can be administered a sphingolipid-like compound, an ARF6 antagonist, or a PIKfyve antagonist. Formulations and medicaments are utilized to formulate therapeutics that can be administered to individuals as pharmaceutically effective salt or in pure form, including, but not limited to, formulations for oral, intravenous, or intramuscular administration.

### STATEMENT REGARDING FEDERALLY SPONSORED RESEARCH OR DEVELOPMENT

This invention was made with Governmental support under NIH NCATS ICTS Award No. TR001414. The government has certain rights in the invention.

### TECHNICAL FIELD

The invention is generally directed to formulations and medicaments and methods for the treatment of metabolic disorders and methods to mitigate mitochondrial fragmentation.

### BACKGROUND

Obesity and type 2 diabetes are diseases that can substantially decrease life expectancy, diminish quality of life and increase healthcare costs. The incidence of obesity and diabetes continues to rise year after year. According to the World Health Organization, an estimated 13% of the world's adult population was obese in 2016, a number that has nearly tripled since 1975. Similarly, according to the American Diabetes Association, in 2002 18.2 million people in the United States, or 6.3 percent of the population, had diabetes. Diabetes was the sixth leading cause of death listed on U.S. death certificates in 2000.

Type 2 diabetes is signified by high levels of glucose in the blood (i.e., hyperglycemia) and obesity is signified with high percentage of stored fat. Typically, when glucose levels are high, the body releases insulin reducing the glucose levels to healthy level. When the body has enough fat stored, adipocytes release leptin to promote satiety and energy expenditure. Overweight individuals and type 2 diabetics, however, eventually become resistant to the elevated levels of insulin and leptin circulating in their blood and fail to respond to medically supplied insulin or leptin. Accordingly, alternative pharmacologic means are needed to treat metabolic disorders, such as hyperglycemia and obesity.

### SUMMARY

In various embodiments, sphingolipid-like molecules are utilized within various drug formulations for treatments of metabolic disorders. In various embodiments, individuals having a metabolic disorder are administered a drug formula inclusive of one or more sphingolipid-like molecules. In various embodiments, metabolic disorders that are treated include (but are not limited to) obesity, metabolic syndrome, hyperglycemia, type 2 diabetes, insulin resistance, leptin resistance, hyperleptinemia, and hepatic steatosis.

In an embodiment, a disorder or condition is treated. A sphingolipid-like compound is administered to a subject having the disorder or condition. The disorder or condition is related to metabolism.

In another embodiment, the sphingolipid-like compound is based on O-benzyl pyrrolidines having the formula:

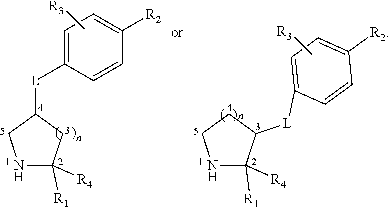

R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof. R₂ is an aliphatic chain (C₆-C₁₀). R₃ is a mono-, di-, tri- or quad-aromatic substituent comprising H, halogen, alkyl, alkoxy, azide (N₃), ether, NO₂, or cyanide (CN). One of R₁ or R₄ is an alcohol (CH₂OH) or H. L is O—CH₂. n is an independently selected integer selected from 1, 2, or 3.

In yet another embodiment, the sphingolipid-like compound is based on diastereomeric 3- and 4-C-aryl pyrrolidines having the formula:

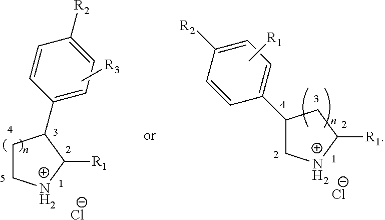

R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof. R₂ is an aliphatic chain (C₆-C₁₀). R₃ is a mono-, di-, tri- or quad-aromatic substituent comprising H, halogen, alkyl, alkoxy, azide (N₃), ether, NO₂, or cyanide (CN). n is an independently selected integer selected from 1, 2, or 3.

In a further embodiment, the sphingolipid-like compound is compound 893 having the formula:

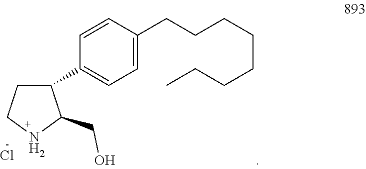 [SMILES: Cl ; CCCCCCCCc1ccc([C@H]2CCN[C@@H]2CO)cc1]

In still yet another embodiment, the sphingolipid-like compound is compound 1090 having the formula:

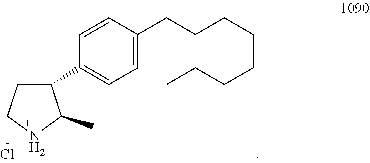 [SMILES: CCCCCCCCc1ccc([C@H]2CCN[C@@H]2C)cc1 ; Cl]

In yet a further embodiment, the sphingolipid-like compound is based on azacycles with an attached heteroaromatic appendage having the formula:

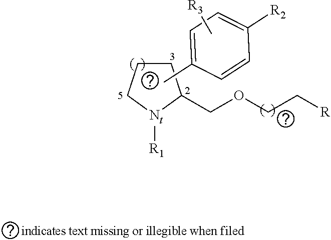

or a pharmaceutically acceptable salt thereof. R is an optionally substituted heteroaromatic moiety such as an optionally substituted pyridazine, optionally substituted pyridine, optionally substituted pyrimidine, phenoxazine, or optionally substituted phenothiazine. R₁ is H, alkyl such as C₁₋₆ alkyl or C₁₋₄ alkyl including methyl, ethyl, propyl, isopropyl, n-butyl, s-butyl, isobutyl, t-butyl, etc, Ac, Boc, guanidine moiety. R₂ is an aliphatic chain comprising 6 to 14 carbons. R₃ is a 1, 2, 3, or 4 substituents, wherein each substituent, independently, is H, halogen, alkyl, alkoxy, N₃, NO₂, and CN. n is independently 1, 2, 3, or 4. m is independently 1 or 2. The phenyl moiety can be attached at any available position of the azacycle core. R is a 1,2-pyridazine having the formula:

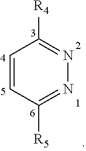

R₄ and R₅ are functional groups independently selected from: alkyl including methyl, optionally substituted aryl (i.e., unsubstituted aryl or substituted aryl) including optionally substituted phenyl, and optionally substituted heteroaryl including optionally substituted pyridine and optionally substituted pyrimidine. The pyridazine moiety is connected to the azacycle at the position 4 or 5 of the pyridazine.

In an even further embodiment, the sphingolipid-like compound is compound 325 having the formula:

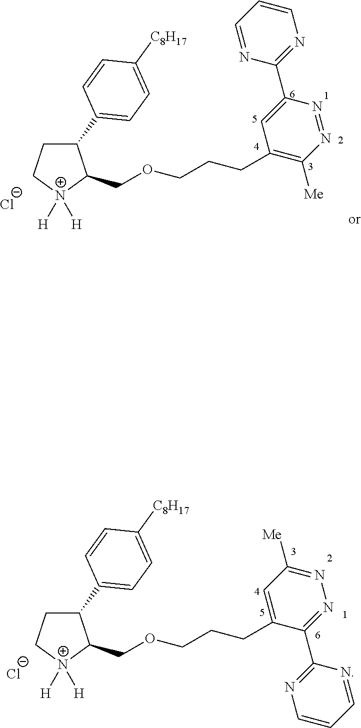

In yet an even further embodiment, the sphingolipid-like compound is based on diastereomeric 2-C-aryl pyrrolidines having the formula:

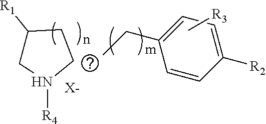

R₁ is a functional group selected from H, an alkyl chain, OH, (CH₂)ₙOH, CHOH-alkyl, CHOH-alkyne, (CH₂)ₙOR′, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof, where R′ is an alkyl, alkene or alkyne. R₂ is an aliphatic chain (C₆-C₁₄). R₃ is a mono-, di-, tri- or tetra-aromatic substituent that includes hydrogen, halogen, alkyl, alkoxy, azide (N₃), ether, NO₂, cyanide (CN), or a combination thereof. R₄ is a functional group selected from H, alkyl including methyl (Me), ester, or acyl. X⁻ is an anion of the suitable acid. n is an independently selected integer selected from 1, 2, or 3. m is an independently selected integer selected from 0, 1 or 2.

In still yet an even further embodiment, the disorder or condition is obesity.

In still yet an even further embodiment, the disease or condition is metabolic syndrome.

In still yet an even further embodiment, the disease or condition is hyperglycemia.

In still yet an even further embodiment, the disease or condition is type 2 diabetes.

In still yet an even further embodiment, the disease or condition is insulin resistance.

In still yet an even further embodiment, the disease or condition is leptin resistance.

In still yet an even further embodiment, the disease or condition is hyperleptinemia.

In still yet an even further embodiment, the disease or condition is hepatic steatosis.

In still yet an even further embodiment, the disease or condition is nonalcoholic steatohepatitis.

In still yet an even further embodiment, the administering of the sphingolipid-like compound reduces the subject's food intake.

In still yet an even further embodiment, the administering of the sphingolipid-like compound decreases weight gain in the subject.

In still yet an even further embodiment, the administering of the sphingolipid-like compound decreases adiposity in the subject.

In still yet an even further embodiment, the administering of the sphingolipid-like compound decreases metabolic dysfunction in the subject.

In still yet an even further embodiment, the administering of the sphingolipid-like compound promotes insulin sensitivity in the subject.

In still yet an even further embodiment, the administering of the sphingolipid-like compound promotes leptin sensitivity in the subject.

In still yet an even further embodiment, the administering of the sphingolipid-like compound improves glucose tolerance.

In still yet an even further embodiment, the administering of the sphingolipid-like compound reduces plasma leptin levels.

In still yet an even further embodiment, the administering of the sphingolipid-like compound reduces plasma insulin levels.

In still yet an even further embodiment, the administering of the sphingolipid-like compound reduces ceramide levels.

In still yet an even further embodiment, the administering of the sphingolipid-like compound increases adiponectin levels.

In still yet an even further embodiment, the administering of the sphingolipid-like compound reduces body fat.

In still yet an even further embodiment, the administering of the sphingolipid-like compound resolves hepatic steatosis in the subject.

In still yet an even further embodiment, the administering of the sphingolipid-like compound resolves steatohepatitis.

In still yet an even further embodiment, the treatment is combined with an FDA-approved or EMA-approved standard of care.

In still yet an even further embodiment, the individual is diagnosed as having the condition or disorder.

In an embodiment, mitochondrial fragmentation is mitigated. A biological cell is contacted with a sphingolipid-like compound, wherein the biological cell is undergoing mitochondrial fragmentation.

In another embodiment, the biological cell is associated a metabolic disorder or condition.

In yet another embodiment, the contacting the biological cell with the sphingolipid-like compound reverses mitochondrial fragmentation.

In a further embodiment, mitochondrial fragmentation is mitigated. A biological cell is contacted with an ARF6 antagonist or a PIKfyve antagonist, wherein the biological cell is undergoing mitochondrial fragmentation.

In still yet another embodiment, the ARF6 antagonist is NAV2729, SecinH3, perphenazine, or a derivative thereof.

In yet a further embodiment, the PIKfyve antagonist is YM201636, APY0201, Apilimod, Late Endosome Trafficking Inhibitor EGA, or a derivative thereof.

In an even further embodiment, the contacting the biological cell with the ARF6 antagonist or the PIKfyve antagonist reverses mitochondrial fragmentation.

In yet an even further embodiment, a disorder or condition is treated. An ARF6 antagonist or a PIKfyve antagonist is administered to a subject having the disorder or condition. The disorder or condition is related to metabolism.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides a strategy for morphometric analysis of mitochondrial networks in vitro, utilized in accordance with various embodiments. Representative images of citrate synthase staining in MEFs treated with vehicle (left panel) or palmitate (PA, right panel) are maximum intensity Z-projections derived from 8 Z-slices. Binarized mitochondrial networks were segmented to tag individual objects. Aspect ratio (tubule width/length) as well as roundness ((4×area)/(π×width)) were measured for all citrate synthase-positive objects on a per cell basis. Skeletonized networks were used to quantify branch length of the tubules. Violin plots show all citrate positive objects in the representative cell (left); the center line is the median and the quartiles define the 25th to 75th percentile. The bar plots show the mean±SEM from the representative image (middle) or from 40 cells from 2 biological replicates (right).

FIG. 2 provides citrate synthase staining in mouse embryonic fibroblasts (MEFs) treated for 3 h with vehicle (1% BSA+ethanol) or palmitate (250 μM) after a 3 h pre-treatment with vehicle (water) or SH-BC-893 (893, 5 μM), generated in accordance with various embodiments.

FIG. 3 provides data graphs of aspect ratio, branch length, and roundness of mitochondria in the MEFs as calculated with ImageJ, generated in accordance with various embodiments. FIG. 3 also provides a data graph depicting C16:0 ceramide (C16:0 CER) levels in MEFs pre-treated for 3 h with vehicle (n=7), SH-BC-893 (5 μM, n=7), myriocin (myr, 10 μM, n=5), or fumonisin B1 (FB1, 30 μM, n=3) then treated with vehicle (1% BSA+ethanol) or palmitate (250 μM) for 3 h, generated in accordance with various embodiments. MEFs were treated with C16:0 CER (100 μM, n=2) for 3 h as a positive control.

FIG. 4 provides data graphs of aspect ratio, branch length, and roundness of mitochondria in the MEFs as calculated with ImageJ, generated in accordance with various embodiments. MEFs were pre-treated with vehicle (water) or SH-BC-893 (5 μM) for 3 h and then treated with vehicle (1% BSA in ethanol) or palmitate (250 μM) for an additional 3 h. Cells were then fixed, stained for citrate synthase. Data for individual citrate synthase-positive objects from 20 cells from 2 biological replicates (3,000-8,000 objects) shown.

FIG. 5 provides data graphs of aspect ratio, branch length, and roundness of mitochondria in the MEFs as calculated with ImageJ, generated in accordance with various embodiments. MEFs were treated with vehicle (ethanol) or C16:0 CER (100 μM) for 3 h after a 3 h pre-treatment with vehicle (water) or SH-BC-893 (5 μM).

FIG. 6 provides data graphs of aspect ratio, branch length, and roundness of mitochondria in the MEFs as calculated with ImageJ, generated in accordance with various embodiments. MEFs were pre-treated with vehicle or 893 (5 μM) for 3 h then treated with vehicle (DMSO) or C2-ceramide (50 μM) for an additional 3 h.

FIG. 7A provides Mander's overlap coefficient for DRP1 and citrate synthase (CS) for the cells in calculated using ImageJ on a per cell basis and a representative DRP1 western blot and quantification of DRP1 levels, generated in accordance with various embodiments. MEFs treated for 3 h with vehicle (1% BSA+ethanol) or palmitate (250 μM) after a 3 h pre-treatment with vehicle (water) or SH-BC-893 (893, 5 μM) and evaluated for DRP1 and citrate synthase co-localization using confocal immunofluorescence microscopy.

FIG. 7B provides citrate synthase staining in mouse embryonic fibroblasts (MEFs) treated for 3 h with vehicle (1% BSA+ethanol) or palmitate (250 μM) after a 3 h pre-treatment with vehicle (water) or NAV-2719 (12.5 μM), generated in accordance with various embodiments.

FIG. 7C provides citrate synthase and Drp1 staining in mouse embryonic fibroblasts (MEFs) treated for 3 h with vehicle (1% BSA+ethanol) or palmitate (250 μM) after a 3 h pre-treatment with vehicle (water) or YM201636 (800 nM), generated in accordance with various embodiments.

FIG. 8 provides data graphs of aspect ratio, branch length, and roundness of mitochondria in the MEFs as calculated with ImageJ, generated in accordance with various embodiments. After a 1 h pre-treatment with vehicle (DMSO), SH-BC-893 (893, 5 μM) or mdivi-1 (50 μM) and M1 (5 μM) for 24 h, MEFs were treated for 3 h with vehicle (ethanol) or C16-CER (100 μM) and stained for citrate synthase.

FIG. 9 provides data graphs of aspect ratio, branch length, and roundness of mitochondria in the A549 cells as calculated with ImageJ, generated in accordance with various embodiments. A549 cells were treated with vehicle (methanol) or 893 (5 μM) for 1 h or leflunomide (50 μM) for 24 h.

FIG. 10 provides data graphs of aspect ratio, branch length, and roundness of mitochondria in the MEFs as calculated with ImageJ, generated in accordance with various embodiments. Control lox-stop-lox (LSL) or KRASG12D MEFs were treated with 893 (5 μM) for 3 h and stained for citrate synthase.

FIG. 11 provides a strategy for morphometric analysis of mitochondrial networks in vivo. Mitochondrial networks in freshly resected livers from mice fed a SD (left panel) or a HFD (right panel) imaged by NADH/NADPH autofluorescence. Images are maximum intensity Z-projections derived from 6 Z-slices. Binarized mitochondrial networks were segmented to tag individual objects. Aspect ratio (tubule width/length) as well as roundness ((4×area)/(π×width)) were measured on a per field basis. In violin plots (left), the center line is the median and the quartiles define the 25th to 75th percentile; data from the representative cell shown. The bar plots show the mean±SEM from the representative cell (middle) or represent per field averages; 8-12 fields of view taken from each of 4 mice per group. The same strategy was applied to quantify hypothalamic mitochondria visualized with a citrate synthase antibody except that 5 fields of view were evaluated from each of 4 mice per group.

FIG. 12 provides a data graph depicting plasma pharmacokinetics in mice after a single dose of 120 mg/kg SH-BC-893 given by gavage (n=3), generated in accordance with various embodiments.

FIG. 13 provides aspect ratio and roundness of mitochondria in the livers of mice, generated in accordance with various embodiments. NADH/NADPH autofluorescence evaluated by confocal microscopy in freshly resected livers from mice that had consumed a SD for 22 weeks or a HFD for 26 weeks after acute treatment with vehicle or 120 mg/kg SH-BC-893 by gavage at ZT8.5. Mice were sacrificed in pairs between ZT13 and ZT17.5.

FIG. 14 provides aspect ratio and roundness of mitochondria in the arcuate nucleus (ARC) of mice, generated in accordance with various embodiments. Mice had consumed a SD for 22 weeks or a HFD for 26 weeks after acute treatment with vehicle or 120 mg/kg SH-BC-893 by gavage at ZT8.5. Mice were sacrificed in pairs between ZT13 and ZT17.5 in alphabetical order.

FIG. 15 provides a table of p-values for FIGS. 16 & 18 to 23 using one-way ANOVA and Tukey's correction, generated in accordance with various embodiments.

FIG. 16 provides a data graph depicting body weight of mice fed a standard diet and gavaged with vehicle (SD, n=10) or fed a high fat diet (HFD) and gavaged with vehicle (n=10), 60 mg/kg (n=9), or 120 mg/kg (n=10) SH-BC-893 on Mondays, Wednesdays, and Fridays beginning on day 49 (arrow), generated in accordance with various embodiments.

FIG. 17 provides data graphs depicting body weight, fat mass, lean mass, body composition as % fat mass, or % lean mass for mice fed a SD (n=10) or HFD (n=40), generated in accordance with various embodiments. In box plots, the center line is the median and the box is delimited by the 25th to 75th percentile, whiskers represent minimum and maximum values.

FIG. 18 provides data graphs depicting percent change of body weight during treatment (days 49-73) for mice described in FIGS. 16 and 17, generated in accordance with various embodiments.

FIG. 19 provides data graphs depicting percent change of fat mass during treatment (days 49-73) and the fat mass on day 73 for mice described in FIGS. 16 and 17, generated in accordance with various embodiments.

FIG. 20 provides data graphs depicting percent change of lean mass during treatment (days 49-73) and the lean mass on day 73 for mice described in FIGS. 16 and 17, generated in accordance with various embodiments.

FIG. 21 provides data graphs depicting body weight and percent change of body weight during treatment (days 49-73) of mice provided with running wheels where indicated and fed a standard diet and gavaged with vehicle (SD, n=10) or fed a high fat diet (HFD) and gavaged with vehicle (n=8), or 120 mg/kg (n=8) SH-BC-893 on Mondays, Wednesdays, and Fridays beginning on day 49 (arrow), generated in accordance with various embodiments.

FIG. 22 provides data graphs depicting percent change of fat mass during treatment (days 49-73) and the fat mass on day 73 for mice described in FIG. 21, generated in accordance with various embodiments.

FIG. 23 provides data graphs depicting percent change of lean mass during treatment (days 49-73) and the lean mass on day 73 for mice described in FIG. 21, generated in accordance with various embodiments.

FIG. 24 provides data graphs depicting ceramide levels in liver or quadriceps muscle from mice fed a SD, HFD, or HFD+120 mg/kg 893 for 73 days; n=4 in all groups, generated in accordance with various embodiments.

FIG. 25 provides a western blot and corresponding data graph depicting insulin-stimulated (100 nm for 15 min) AKT activation in 3T3-L1 adipocytes pre-treated with C2-ceramide (50 or 100 μM) or SH-BC-893 (5 or 10 μM) for 3 h, generated in accordance with various embodiments.

FIG. 26 provides a data graph depicting insulin-stimulated 2-deoxyglucose uptake in 3T3-L1 adipocytes after 3 h of treatment with vehicle (n=5), SH-BC-893 (10 μM, n=5) or MK-2206 (2 μM, n=4), generated in accordance with various embodiments. FIG. 26 further provides a data graph depicting 2-deoxyglucose uptake in mouse embryonic fibroblasts after a 3 h treatment with vehicle (n=4) or 893 (5 (n=4) or 10 (n=3) μM), generated in accordance with various embodiments.

FIG. 27 provides a data graph depicting fasting blood glucose level from mice in FIG. 16 after 25 d of treatment. SD+vehicle and HFD+vehicle (n=6), HFD+60 or 120 mg/kg SH-BC-893 (n=4), generated in accordance with various embodiments. FIG. 27 further provides data graphs depicting blood glucose levels or area under the curve (AUC) during an oral glucose tolerance test performed in these mice, generated in accordance with various embodiments.

FIG. 28 provides data graphs depicting respiratory exchange ratio (RER) of mice fed a SD (n=8) for 10-12 weeks and then treated with vehicle or 120 mg/kg SH-BC-893 p.o. at ZT8.5 on days 1 (first exposure) and 3, generated in accordance with various embodiments. The means of 4 measurements over 108 minutes or average value over the dark (ZT12-ZT24) or light (ZT0-ZT12) cycle shown. Treatment indicated with arrows or *.

FIG. 29 provides data graphs depicting respiratory exchange ratio (RER) of mice fed a HFD (n=6-7) for 10-12 weeks and then treated with vehicle or 120 mg/kg SH-BC-893 p.o. at ZT8.5 on days 1 (first exposure) and 3, generated in accordance with various embodiments. The means of 4 measurements over 108 minutes or average value over the dark (ZT12-ZT24) or light (ZT0-ZT12) cycle shown. Treatment indicated with arrows or *.

FIG. 30 provides a data graph depicting leptin levels in serum of HFD-fed mice for 4 weeks treated with water (n=7) or 120 mg/kg 893 (n=8) at ZT8.5, generated in accordance with various embodiments.

FIG. 31 provides data graphs depicting food intake during the indirect calorimetry studies in FIG. 29 shown as the means of 4 measurements taken over 108 min or averaged from ZT12-ZT24, generated in accordance with various embodiments. Mice were fed a SD and treated with vehicle (n=6-8) or 120 mg/kg SH-BC-893 (n=5-8).

FIG. 32 provides data graphs depicting food intake during the indirect calorimetry studies shown in FIG. 30 as the means of 4 measurements taken over 108 min or averaged from ZT12-ZT24, generated in accordance with various embodiments. Mice were fed a HFD and treated with vehicle (n=6-8) or 120 mg/kg SH-BC-893 (n=5-8).

FIG. 33 provides data graphs depicting food intake and body weight change from ZT12-ZT24 in mice fed a SD for 10 weeks (n=8), a HFD for 10 weeks (n=7), or a HFD for 22 weeks (n=8) and treated once at ZT8.5 with vehicle or 120 mg/kg SH-BC-893 by gavage, generated in accordance with various embodiments

FIG. 34 provides a data graph depicting average RER between ZT12-ZT24 in mice fed a HFD for 22 weeks and then treated with vehicle (n=8), 120 mg/kg SH-BC-893 (n=8), or pair fed the average amount of food eaten during this period by SH-BC-893-treated mice (n=8), generated in accordance with various embodiments.

FIG. 35 provides data graphs depicting food intake and body weight change from ZT8.5-ZT2.5 (18 h) in 18 week-old mice fed a 16% kcal from fat chow diet and treated with vehicle (saline) or 2 mg/kg leptin i.p. at ZT11.5 and with vehicle (water) or 120 mg/kg SH-BC-893 by gavage at ZT8.5; all groups, n=7, generated in accordance with various embodiments.

FIG. 36 provides a data graph depicting body weight of wild type C57BL/6J mice fed a HFD for 24 weeks (n=8) or 10 week old ob/ob mice (n=9) fed a 16% kcal from fat chow diet prior to treatment with SH-BC-893, generated in accordance with various embodiments. FIG. 36 further provides data graphs depicting food intake or body weight change from ZT8.5-ZT2.5 (18 h) in the wild type C57BL/6J mice fed a HFD for 24 weeks (n=8) or 10 week old ob/ob mice (n=9) fed a 16% kcal from fat chow diet after a single oral dose of vehicle or 120 mg/kg SH-BC-893, generated in accordance with various embodiments.

FIG. 37 provides data graphs depicting body weight or percent change in body weight in ob/ob mice treated Monday, Wednesday, and Friday with vehicle (n=4) or 120 mg/kg SH-BC-893 (n=4) by gavage for 2 weeks, generated in accordance with various embodiments.

FIG. 38 provides a data graph depicting cumulative food intake in ob/ob mice treated Monday, Wednesday, and Friday with vehicle (n=4) or 120 mg/kg SH-BC-893 (n=4) by gavage for 2 weeks, generated in accordance with various embodiments. FIG. 38 further provides a data graph depicting oral glucose tolerance test performed in ob/ob mice treated Monday, Wednesday, and Friday with vehicle (n=4) or 120 mg/kg SH-BC-893 (n=4) by gavage for 2 weeks, generated in accordance with various embodiments. The test was performed 14 d after the initiation of treatment. Open circles indicate where some blood glucose values exceeded the limit of detection (>600 mg/dL) and were assigned a value of 600 mg/dL.

FIG. 39 provides data graphs depicting aspect ratio and roundness of mitochondria in freshly resected livers from 12 week old ob/ob mice treated with vehicle or 120 mg/kg SH-BC-893 by gavage at ZT8.5, generated in accordance with various embodiments.

### DETAILED DESCRIPTION

Turning now to the drawings and data, sphingolipid-like molecules, medicaments formed from these molecules, and methods for the treatment of metabolic disorders using such therapeutics, in accordance with various embodiments, are disclosed. In certain embodiments, a sphingolipid-like molecule is utilized to mitigate mitochondrial fragmentation within a biological cell, especially within cells associated with a metabolic disorder. In certain embodiments, a sphingolipid-like molecule is utilized in therapeutic to treat a metabolic disorder. In certain embodiments, a therapeutic contains a therapeutically effective dose of one or more sphingolipid-like molecule compounds, present either as pharmaceutically effective salt or in pure form. In certain embodiments, an individual having a metabolic disorder is administered a therapeutic incorporating one or more sphingolipid-like molecules. In certain embodiments, metabolic disorders targeted with sphingolipid-like molecules include (but are not limited to) obesity, hyperglycemia, insulin resistance, leptin resistance, hyperleptinemia, and hepatic steatosis. In certain embodiments, therapeutics incorporating one or more sphingolipid-like molecules reduce a subject's food intake, reduce weight gain, improve insulin sensitivity, improve glucose tolerance, improve leptin sensitivity, reduce plasma leptin levels, reduce plasma insulin levels, reduce ceramide levels, increase adiponectin levels, decrease adiposity, decrease metabolic dysfunction, reduce body fat, resolve hepatic steatosis and/or resolves steatohepatitis. Various embodiments utilize various formulations, including (but not limited to) formulations for oral, intravenous, or intramuscular administration.

Various embodiments of therapeutics can incorporate one or more of any appropriate sphingolipid-like molecule compounds. In some embodiments, sphingolipid-like molecules are based on O-benzyl azacycles. In some embodiments, sphingolipid-like molecules are based on 2-, 3-, and 4-C-aryl azacycles. In some embodiments, sphingolipid-like molecules are based on azacycles with heteroaromatic appendage.

In certain embodiments, an ARF6 antagonist or a PIKfyve antagonist is utilized to mitigate mitochondrial fragmentation within a biological cell, especially within cells associated with a metabolic disorder. In certain embodiments, an ARF6 antagonist or a PIKfyve antagonist is utilized in therapeutic to treat a metabolic disorder. In certain embodiments, a therapeutic contains a therapeutically effective dose of one or more ARF6 antagonist or PIKfyve antagonist compounds, present either as pharmaceutically effective salt or in pure form. In certain embodiments, an individual having a metabolic disorder is administered a therapeutic incorporating one or more ARF6 antagonists or PIKfyve antagonists. In certain embodiments, metabolic disorders targeted with ARF6 antagonists or PIKfyve antagonists include (but are not limited to) obesity, hyperglycemia, insulin resistance, leptin resistance, hyperleptinemia, and hepatic steatosis. In certain embodiments, therapeutics incorporating one or more ARF6 antagonists or PIKfyve antagonists reduce a subject's food intake, reduce weight gain, improve insulin sensitivity, improve glucose tolerance, improve leptin sensitivity, reduce plasma leptin levels, reduce plasma insulin levels, reduce ceramide levels, increase adiponectin levels, decrease adiposity, decrease metabolic dysfunction, reduce body fat, resolve hepatic steatosis and/or resolves steatohepatitis. Various embodiments utilize various formulations, including (but not limited to) formulations for oral, intravenous, or intramuscular administration.

High fat diets contribute to various metabolic diseases via altering mitochondrial structure, causing fragmentation, thus reducing their ability to meet the bioenergetic demands of various tissues/organs in the body. Mitochondrial fragmentation has been linked to a reduced response to leptin and insulin and to an increased production of leptin that contributes to obesity. It has been found that sphingolipid-like compounds, such as those compounds described herein, inhibit and reverse mitochondrial fragmentation in mice and in mouse and human cells. It was further shown that sphingolipid-like compounds reduce food intake, decrease weight gain, decrease adiposity, decrease metabolic dysfunction, resolve hepatic steatosis, reduce plasma leptin levels, reduce plasma insulin levels, reduce ceramide levels, and promote insulin and leptin sensitivity in mice on high fat diets. Based on these findings, and in accordance with various embodiments, sphingolipid-like molecules are utilized to treat metabolic disorders associated with high fat diets, obesity, hyperglycemia, insulin resistance, leptin resistance, hyperleptinemia, and/or hepatic steatosis.

Furthermore, sphingolipid-like compounds are antagonists of the cytosolic enzymes ADP Ribosylation Factor 6 (ARF6) and Phosphoinositide Kinase, FYVE-Type Zinc Finger Containing (PIKfyve), which are involved in endosome recycling and endosome fusion with lysomes (B. T. Finicle, et al., J Cell Sci. 131:jcs213314, 2018; and S. M. Kim, et al., J Clin Invest. 126:4088-4102, 2016; the disclosures of which are incorporated herein by reference). ARF6 induces endocytic vesicles to be recycled, fusing with the plasma membrane. PIKfyve promotes endosome-lysosome fusion. It has been shown that inhibitors of these proteins also inhibit and reverse mitochondrial fragmentation in mouse embryonic fibroblasts (MEFs) treated with palmitate. Based on these findings, and in accordance with various embodiments, antagonists of ARF6 and PIKfyve are utilized to treat metabolic disorders associated with high fat diets, obesity, hyperglycemia, insulin resistance, leptin resistance, hyperleptinemia, and/or hepatic steatosis.

### Definitions

For the purposes of this description, the following definitions are used, unless otherwise described.

“Pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water; sterile saline; cremophor; or sterile buffer solution.

“Pharmaceutically acceptable salts” means physiologically and pharmaceutically acceptable salts of compounds, such as antiviral compounds, i.e., salts that retain the desired biological activity of the parent compound and do not impart undesired toxicological effects thereto.

“Pharmaceutical composition” means a mixture of substances suitable for administering to a subject. For example, a pharmaceutical composition may comprise an antiviral compound and a sterile aqueous solution.

“Prodrug” means a therapeutic agent in a form outside the body that is converted to a different form within the body or cells thereof. Typically conversion of a prodrug within the body is facilitated by the action of an enzymes (e.g., endogenous or viral enzyme) or chemicals present in cells or tissues and/or by physiologic conditions.

“Metabolic disorder” means an abnormality in body metabolism and includes (but is not limited to) obesity, hyperglycemia, insulin resistance, leptin resistance, hyperleptinemia, and hepatic steatosis (e.g., nonalcoholic hepatic steatosis (NASH)). Hyperglycemia is indicated by elevate glucose in the blood and includes conditions of pre-diabetes and type 2 diabetes.

### Terms of Art

“Acyl” means a —R—C═O group.

“Alcohol” means a compound with an —OH group bonded to a saturated, alkane-like compound, (ROH).

“Alkyl” refers to the partial structure that remains when a hydrogen atom is removed from an alkane.

“Alkane” means a compound of carbon and hydrogen that contains only single bonds.

“Alkene” refers to a hydrocarbon that contains a carbon-carbon double bond, R₂C═CR₂.

“Alkyne” refers to a hydrocarbon structure that contains a carbon-carbon triple bond.

“Alkoxy” refers to a portion of a molecular structure featuring an alkyl group bonded to an oxygen atom.

“Aryl” refers to any functional group or substituent derived from an aromatic ring.

“Amine” molecules are compounds containing one or more organic substituents bonded to a nitrogen atom, RNH₂, R₂NH, or R₃N.

“Amino acid” refers to a difunctional compound with an amino group on the carbon atom next to the carboxyl group, RCH(NH₂)CO₂H.

“Azide” refers to N₃.

“Cyanide” refers to CN.

“Ester” is a compound containing the —CO₂R functional group.

“Ether” refers to a compound that has two organic substituents bonded to the same oxygen atom, i.e., R—O—R′.

“Halogen” or “halo” means fluoro (F), chloro (Cl), bromo (Br), or iodo (I).

“Hydrocarbon” means an organic chemical compound that consists entirely of the elements carbon (C) and hydrogen (H).

“Phosphate”, “phosphonate”, or “PO” means a compound containing the elements phosphorous (P) and oxygen (O).

“R” in the molecular formula above and throughout are meant to indicate any suitable organic molecule.

### Compounds for Mitigating Mitochondrial Fragmentation and Treatment of Metabolic Disorders

In certain embodiments, various compounds are used for treatment of metabolic disorders. In certain embodiments, various compounds are administered to a subject having a metabolic disorder. Subjects include in vivo, ex vivo, and in vitro subjects. Accordingly, subjects include (but are not limited to) animals, harvested organ tissues, organoids, and cell lines. Animals include (but are not limited to) humans and animal models (e.g., mice). In some embodiments, cell lines, organ tissues, and/or organoids are derived from tissue extracted from a human or animal model. As discussed herein, mitochondrial fragmentation contributes to abnormal metabolism, which is present in subjects having a metabolic disorders. Compounds for treatment of metabolic disorders include (but are not limited to) ARF6 antagonists, PIKfyve antagonists, and sphingolipid-like compounds. ARF6 antagonists include (but are not limited to) sphingolipid-like compounds, NAV2729, SecinH3, perphenazine, and derivatives thereof. Numerous ARF6 antagonists are described in the literature and can be utilized in certain embodiments as described herein (see B. T. Finicle, et al., J Cell Sci. 131:jcs213314, 2018; J. H. Yoo, et al., Cancer Cell. 29:889-904, 2016; and M. Hafner, et al., Nature. 444:941-944, 2006; the disclosures of which are incorporated herein by reference). PIKfyve antagonists include (but are not limited to) sphingolipid-like compounds, YM201636, APY0201, Apilimod, Late Endosome Trafficking Inhibitor EGA, and derivatives thereof. Numerous PIKfyve antagonists are described in the literature and can be utilized in certain embodiments as described herein (see S. M. Kim, et al., J Clin Invest. 2016; 126(11):4088-4102; H. B. Jefferies, et al., EMBO Rep. 9:164-170, 2008; and X. Cai, et al., Chem Biol. 20:912-921, 2013; the disclosures of which are incorporated by reference). Sphingolipid-like compounds include (but are not limited to) sphingolipids, sphingolipid-like compound 893, sphingolipid-like compound 1090, and sphingolipid-like compound 325.

In certain embodiments, a compound for treatment of a metabolic disorder is utilized at concentration between 1 nM to 100 μM. In certain embodiments a compound is utilized at concentration on the order of less than 1 nM, 1 nM, 10 nM, 100 nM, 1 μM, 10 μM, 100 μM, or greater than 100 μM.

I. Sphingolipid-Like Compounds

A. Sphingolipid-Like Compounds Based on O-Benzyl Azacycles

In certain embodiments, a sphingolipid-like compound is based on O-benzyl azacycles. In certain embodiments, a sphingolipid-like compound is of formula:

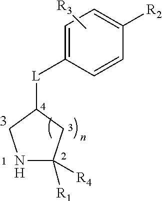

R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof;

R₂ is an aliphatic chain (C₆-C₁₀);

R₃ is a mono-, di-, tri- or quad-aromatic substituent comprising H, halogen, alkyl, alkoxy, azide (N₃), ether, NO₂, or cyanide (CN);

One of R₁ R₄ is an alcohol (CH₂OH) or H;

L is O—CH₂; and

n is an independently selected integer selected from 1, 2, or 3.

In certain embodiments of O-benzyl azacycles, the O-benzyl group can be moved to position 4 (shown above) or 3 as shown below:

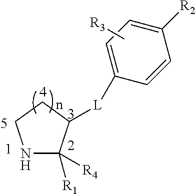

In certain embodiments, alkyl, CH₂OH, or (CH₂)ₙOH groups can be added to position 5.

In certain embodiments, one of R₁ or R₄ is an alkyl having 1 to 6 carbons.

It will be understood that compounds described herein may exist as stereoisomers, including phosphate, phosphonates, enantiomers, diastereomers, cis, trans, syn, anti, solvates (including hydrates), tautomers, and mixtures thereof.

In many embodiments where the compound is a phosphate or phosphonate, R₁ may be, for example, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof.

B. Sphingolipid-Like Compounds Based on 3- and 4-C-Aryl Azacycles

In certain embodiments, a sphingolipid-like compound is based on diastereomeric 3- and 4-C-aryl azacycles. In certain embodiments, a sphingolipid-like compound is of formula:

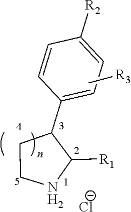

R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof;

R₂ is an aliphatic chain (C₆-C₁₄);

R₃ is a mono-, di-, tri- or tetra-aromatic substituent comprising hydrogen, halogen, alkyl, alkoxy, azide (N₃), ether, NO₂, or cyanide (CN); and

n is an independently selected integer selected from 1, 2, or 3.

In certain embodiments of diastereomeric 3- and 4-C-aryl azacycles, the C-aryl group can be moved to position 3 (shown above) or 4 as shown below:

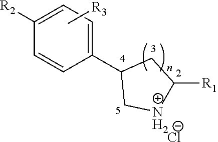

In certain embodiments, alkyl, CH₂OH, or (CH₂)ₙOH groups can be added to position 5.

In certain embodiments, R₂ is an unsaturated hydrocarbon chain.

In certain embodiments, the R₁ is an alkyl having 1 to 6 carbons.

It will be understood that compounds described herein may exist as stereoisomers, including phosphate, phosphonates, enantiomers, diastereomers, cis, trans, syn, anti, solvates (including hydrates), tautomers, and mixtures thereof.

In certain embodiments where the compound is a phosphate or phosphonate, R₁ may be, for example, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof.

In certain embodiments, a sphingolipid-like compound is compound 893, having the formula:

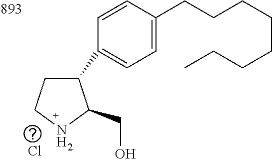 [SMILES: Cl ; CCCCCCCCc1ccc([C@H]2CCN[C@@H]2CO)cc1]

In certain embodiments, a sphingolipid-like compound is compound 1090, having the formula:

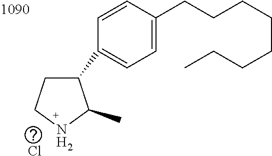 [SMILES: CCCCCCCCc1ccc([C@H]2CCN[C@@H]2C)cc1 ; Cl]

C. Sphingolipid-Like Compounds Based on Azacycles with Heteroaromatic Appendage

In certain embodiments, an antiviral compound is based on azacycles with an attached heteroaromatic appendage. In certain embodiments, a sphingolipid-like compound is of formula:

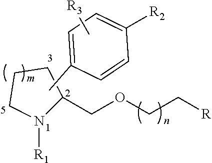

or a pharmaceutically acceptable salt thereof,

R is an optionally substituted heteroaromatic moiety such as an optionally substituted pyridazine, optionally substituted pyridine, optionally substituted pyrimidine, phenoxazine, or optionally substituted phenothiazine.

R₁ is H, alkyl such as C₁₋₆ alkyl or C₁₋₄ alkyl including methyl, ethyl, propyl, isopropyl, n-butyl, s-butyl, isobutyl, t-butyl, etc, Ac, Boc, guanidine moiety.

R₂ is an aliphatic chain comprising 6 to 14 carbons.

R₃ is a 1, 2, 3, or 4 substituents, wherein each substituent, independently, is H, halogen, alkyl, alkoxy, N₃, NO₂, and CN.

n is independently 1, 2, 3, or 4.

m is independently 1 or 2.

The phenyl moiety can be attached at any available position of the azacycle core.

In some embodiments, R₂ is an unsaturated hydrocarbon chain.

In some embodiments, R₂ is C₆₋₁₄ alkyl, C₆₋₁₀ alkyl, C₇₋₉ alkyl, C₆H₁₃, C₇H₁₅, C₈H₁₇, C₉H₁₉, C₁₀H₂₁, C₁₁H₂₃, C₁₂H₂₅, C₁₃H₂₇, or C₁₄H₂₉.

In some embodiments R₃ is H.

In some embodiments, n is 1. In some embodiments, n is 2. In some embodiments, n is 3. In some embodiments, n is 4.

In some embodiments, m is 1. In some embodiments, m is 2.

In some embodiments, the R₂ and R₃ substituents can have different combinations around the phenyl ring with regard to their position.

In some embodiments, the R₁ is an alkyl having 1 to 6 carbons.

It will be understood that compounds described herein may exist as stereoisomers, including enantiomers, diastereomers, cis, trans, syn, anti, solvates (including hydrates), tautomers, and mixtures thereof, are contemplated in the compounds described herein.

In some embodiments, R is a 1,2-pyridazine having the formula:

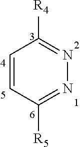

R₄ and R₅ are functional groups independently selected from: alkyl including methyl, optionally substituted aryl (i.e., unsubstituted aryl or substituted aryl) including optionally substituted phenyl, and optionally substituted heteroaryl including optionally substituted pyridine and optionally substituted pyrimidine.

The pyridazine moiety is connected to the azacycle at the position 4 or 5 of the pyridazine.

In some embodiments, any substituents of R₄ and R₅, if present, are independently halogen including F, alkyl, terminal alkyne, and azide.

In some embodiments, R₄ is C₁₋₆ alkyl, such as CH₃, C₂ alkyl, C₃ alkyl, C₄ alkyl, C₅ alkyl, or C₆ alkyl; unsubstituted aryl or substituted aryl, including unsubstituted phenyl, or phenyl having 1, 2, 3, 4, or 5 substituents; unsubstituted heteroaryl or substituted heteroaryl, including unsubstituted pyridine or pyridine having 1, 2, 3, or 4 substituents, or unsubstituted pyrimidine or pyrimidine having 1, 2, or 3 substituents. Any substituent may be used in the substituted aryl (e.g., substituted phenyl) or substituted heteroaryl (e.g., substituted pyridine or substituted pyrimidine). For example, the substituents of the substituted aryl or substituted heteroaryl may independently be, halo (such as F, Cl, Br, I), C₁₋₆ alkyl (such as CH₃, C₂ alkyl, C₃ alkyl, C₄ alkyl, C₅ alkyl, C₆ alkyl), or X—R^{a}, wherein X is O, —C(═O)—, —NHC(═O)—, or —C(═O)NH—, and R^{a} is C₁₋₆ alkyl (such as CH₃, C₂ alkyl, C₃ alkyl, C₄ alkyl, C₅ alkyl, C₆ alkyl), C₂₋₆ alkenyl (such as —CH═CH₂, —CH₂CH═CH₂, —CH₂CH₂CH═CH₂, —CH₂CH₂CH₂CH═CH₂, —CH₂CH₂CH₂CH₂CH═CH₂, etc.), or C₂₋₆ alkynyl (such as —CH═CH₂, —CH₂CH═CH₂, —CH₂CH₂CH═CH₂, —CH₂CH₂CH₂CH═CH₂, —CH₂CH₂CH₂CH₂CH═CH₂, etc.); or azide.

In some embodiments, R₅ is C₁₋₆ alkyl, such as CH₃, C₂ alkyl, C₃ alkyl, C₄ alkyl, C₅ alkyl, or C₆ alkyl; unsubstituted aryl or substituted aryl, including unsubstituted phenyl, or phenyl having 1, 2, 3, 4, or 5 substituents; unsubstituted heteroaryl or substituted heteroaryl, including unsubstituted pyridine or pyridine having 1, 2, 3, or 4 substituents, or unsubstituted pyrimidine or pyrimidine having 1, 2, or 3 substituents. Any substituent may be used in the substituted aryl (e.g., substituted phenyl) or substituted heteroaryl (e.g., substituted pyridine or substituted pyrimidine). For example, the substituents of the substituted aryl or substituted heteroaryl may independently be, halo (such as F, Cl, Br, I), C₁₋₆ alkyl (such as CH₃, C₂ alkyl, C₃ alkyl, C₄ alkyl, C₅ alkyl, C₆ alkyl), or X—R^{a}, wherein X is O, —C(═O)—, —NHC(═O)—, or —C(═O)NH—, and R^{a} is C₁₋₆ alkyl (such as CH₃, C₂ alkyl, C₃ alkyl, C₄ alkyl, C₅ alkyl, C₆ alkyl), C₂₋₆ alkenyl (such as —CH═CH₂, —CH₂CH═CH₂, —CH₂CH₂CH═CH₂, —CH₂CH₂CH₂CH═CH₂, —CH₂CH₂CH₂CH₂CH═CH₂, etc.), or C₂₋₆ alkynyl (such as —CH═CH₂, —CH₂CH═CH₂, —CH₂CH₂CH═CH₂, —CH₂CH₂CH₂CH═CH₂, —CH₂CH₂CH₂CH₂CH═CH₂, etc.); or azide.

In some embodiments, R₄ and R₅ are the same functional group.

In some embodiments, R₄ and R₅ are different functional groups.

In some embodiments, R₄ is C₁₋₆ alkyl, such as methyl, and R₅ is optionally substituted phenyl.

In some embodiments, R₄ is C₁₋₆ alkyl, such as methyl, and R₅ is optionally substituted pyridine.

In some embodiments, R₄ is C₁₋₆ alkyl, such as methyl, and R₅ is optionally substituted pyrimidine.

In some embodiments, R₄ is optionally substituted pyridine and R₅ is optionally substituted pyridine.

In some embodiments, R₄ is optionally substituted phenyl and R₅ is optionally substituted phenyl.

In some embodiments, R₄ is optionally substituted phenyl and R₅ is optionally substituted pyrimidine.

In some embodiments, R is an optionally substituted phenoxazine or an optionally substituted phenothiazine, such as phenoxazine or phenthiazine having the formula:

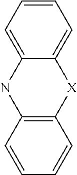

which may additionally have substituents on any available ring position.

X is selected from: O and S.

R is attached to the azacycle via R's nitrogen.

Substituents of R may independently include halogen, alkyl (e.g., C₁₋₆ alkyl, such as CH₃, C₂ alkyl, C₃ alkyl, C₄ alkyl, C₅ alkyl, or C₆ alkyl), alkoxy (e.g., C₁₋₆ alkoxy, such as —OCH₃, C₂ alkoxy, C₃ alkoxy, C₄ alkoxy, C₅ alkoxy, or C₆ alkoxy), N₃, NO₂, and CN.

It will be understood that compounds described herein may exist as stereoisomers, including phosphate, phosphonates, enantiomers, diastereomers, cis, trans, syn, anti, solvates (including hydrates), tautomers, and mixtures thereof.

In certain embodiments, a sphingolipid-like compound is compound 325, having the formula:

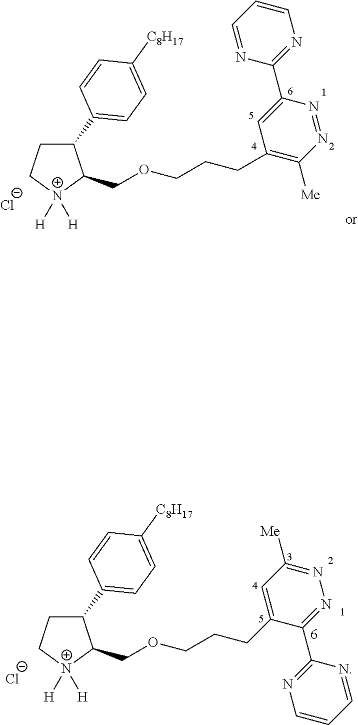

D. Sphingolipid-Like Compounds Based on 2-C-Aryl Azacycles

In certain embodiments, a sphingolipid-like compound is based on diastereomeric 2-C-aryl azacycles. In certain embodiments, a sphingolipid-like compound is of formula:

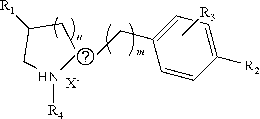

R₁ is a functional group selected from H, an alkyl chain, OH, (CH₂)ₙOH, CHOH-alkyl, CHOH-alkyne, (CH₂)ₙOR′, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof, where R′ is an alkyl, alkene or alkyne.

R₂ is an aliphatic chain (C₆-C₁₄).

R₃ is a mono-, di-, tri- or tetra-aromatic substituent that includes hydrogen, halogen, alkyl, alkoxy, azide (N₃), ether, NO₂, cyanide (CN), or a combination thereof.

R₄ is a functional group selected from H, alkyl including methyl (Me), ester, or acyl.

X⁻ is an anion of the suitable acid.

n is an independently selected integer selected from 1, 2, or 3.

m is an independently selected integer selected from 0, 1 or 2.

The molecule can include an optional functional group of the azacycle's substituent selected from the following:

a polar group in the alpha, beta or gamma position with regard to the azacycle selected from carbonyls (C═O) and alcohols (CHOH);

*a cyclic carbon chain extending from the alpha, beta or gamma positions with regard to the azacycle back to the N of the azacycle, and

a combination thereof.

In some embodiments, R₁ is H, OH, CH₂OH, OPO(OH)₂. In some embodiments, R₁ is H. In some embodiments, R₁ is OH. In some embodiments, R₁ is CH₂OH. In some embodiments, R₁ is OPO(OH)₂.

In some embodiments, R₂ is C₆₋₁₄ alkyl, C₆₋₁₀ alkyl, C₇₋₉ alkyl, C₆H₁₃, C₇H₁₅, C₈H₁₇, C₉H₁₉, C₁₀H₂₁, C₁₁H₂₃, C₁₂H₂₅, C₁₃H₂₇, or C₁₄H₂₉. In some embodiments, R₂ is C₈H₁₇.

In some embodiments R₃ is H.

In some embodiments, n is 1.

In some embodiments m is 0. In some embodiments, m is 1. In some embodiments, m is 2. In some embodiments, m is 3.

In some embodiments, the linking group connecting the phenyl ring to the azacycle is C(═O), CH₂C(═O), C(═O)CH₂, CH₂CH₂C(═O), CH₂, CH₂CH₂, CH₂C(OCH₃)H, or CHOHCH₂. In some embodiments, the linking group connecting the phenyl ring to the azacycle is C(═O). In some embodiments, the linking group connecting the phenyl ring to the azacycle is CH₂C(═O). In some embodiments, the linking group connecting the phenyl ring to the azacycle is C(═O)CH₂. In some embodiments, the linking group connecting the phenyl ring to the azacycle is CH₂CH₂C(═O). In some embodiments, the linking group connecting the phenyl ring to the azacycle is CH₂. In some embodiments, the linking group connecting the phenyl ring to the azacycle is CH₂CH₂. In some embodiments, the linking group connecting the phenyl ring to the azacycle is CH₂C(OCH₃)H. In some embodiments, the linking group connecting the phenyl ring to the azacycle is CHOHCH₂.

In some embodiments, the linking group connecting the phenyl ring to the azacycle includes a cyclic carbon chain extending from the alpha, beta or gamma positions with regard to the azacycle back to the N of the azacycle, so that the azaycle with the linking group form an optionally substituted bicyclic ring of the formula:

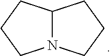

In some embodiments, R₄ is H. In some embodiments, R₄ is C₁₋₆ alkyl, such as CH₃, C₂H₅, C₃H₇, C₄H₉, C₅H₁₁, C₆H₁₃, C₁₋₃ alkyl, etc., C₁₋₆ acyl, or C₁₋₆ ester. In some embodiments, R₄ is methyl.

In still other embodiments, the R₂ and R₃ substituents can have different combinations around the phenyl ring with regard to their position.

In still other embodiments, R₂ is an unsaturated hydrocarbon chain.

In still other embodiments, the R₁ is an alkyl having 1 to 6 carbons.

It will be understood that compounds described herein may exist as stereoisomers, including phosphate, phosphonates, enantiomers, diastereomers, cis, trans, syn, anti, solvates (including hydrates), tautomers, and mixtures thereof.

E. Pharmaceutical Salts of Sphingolipid-Like Compounds

Certain sphingolipid-like compounds can also be related to pharmaceutically acceptable salts. A “pharmaceutically acceptable salt” retains the desirable biological activity of the compound without undesired toxicological effects. Salts can be salts with a suitable acid, including, but not limited to, hydrochloric acid, hydrobromic acid, sulfuric acid, phosphoric acid, nitric acid and the like; acetic acid, oxalic acid, tartaric acid, succinic acid, malic acid, benzoic acid, pamoic acid, alginic acid, methanesulfonic acid, naphthalenesulphonic acid, and the like. Also, incorporated cations can include ammonium, sodium, potassium, lithium, zinc, copper, barium, bismuth, calcium, and the like; or organic cations such as tetraalkylammonium and trialkylammonium cations. Also useful are combinations of acidic and cationic salts. Included are salts of other acids and/or cations, such as salts with trifluoroacetic acid, chloroacetic acid, and trichloroacetic acid.

### Certain Pharmaceutical Compositions

In certain embodiments, the present disclosure provides pharmaceutical compositions comprising one or more compounds or a salt thereof for treatment of a metabolic disorder. In various embodiments, compounds utilized in a pharmaceutical formulation is a sphingolipid-like molecule, an ARF6 antagonist, and/or a PIKfyve antagonist. In certain embodiments, the pharmaceutical composition includes a suitable pharmaceutically acceptable diluent or carrier. In certain embodiments, a pharmaceutical composition comprises a sterile saline solution and one or more compounds. In certain embodiments, such pharmaceutical composition consists of a sterile saline solution and one or more compounds. In certain embodiments, the sterile saline is pharmaceutical grade saline. In certain embodiments, a pharmaceutical composition comprises sterile water and one or more compounds. In certain embodiments, the water is pharmaceutical grade water. In certain embodiments, a pharmaceutical composition comprises phosphate-buffered saline (PBS) and one or more compounds. In certain embodiments, the PBS is pharmaceutical grade PBS.

In certain embodiments, pharmaceutical compositions comprise one or more compounds and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, compounds are admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising a compound for treatment of a metabolic disorder encompass any pharmaceutically acceptable salts of the compound, esters of the antisense compound, or salts of such esters. In certain embodiments, pharmaceutical compositions comprising one or more compounds, upon administration to an animal, including a human, are capable of providing (directly or indirectly) the biologically active metabolite or residue thereof. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to a compound, wherein the conjugate group is cleaved by endogenous nucleases within the body.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethyl sulfoxide (DMSO) are used.

In certain embodiments, pharmaceutical compositions comprise a co-solvent system. Certain such co-solvent systems comprise, for example, benzyl alcohol, a nonpolar surfactant, a water-miscible organic polymer, and an aqueous phase. In certain embodiments, co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose. In certain embodiments, dimethyl sulfoxide (DMSO) is utilized as a co-solvent. In certain embodiments, cremophor (or cremophor EL) is utilized as a co-solvent.

In certain embodiments, pharmaceutical compositions comprise one or more compounds that increase bioavailability. For example, 2-hydroxypropyl-beta-cyclodextrin can be utilized in pharmaceutical compositions and may increase bioavailability. In certain embodiment, DMSO, cremophor and 2-hydroxypropyl-beta-cyclodextrin is utilized to increase bioavailability of various sphingolipid-like compounds.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

In certain embodiments, a pharmaceutical composition is administered in a therapeutically effective amount as part of a course of treatment. As used in this context, to “treat” means to ameliorate or prevent at least one symptom of the disorder to be treated or to provide a beneficial physiological effect. A therapeutically effective amount can be an amount sufficient to prevent reduce, ameliorate or eliminate the symptoms of diseases or pathological conditions susceptible to such treatment. In certain embodiments, a therapeutically effective amount is an amount sufficient to improve insulin sensitivity, improve glucose tolerance, improve leptin sensitivity, reduce leptin levels, increase adiponectin levels, and/or reduce body fat.

Dosage, toxicity and therapeutic efficacy of a pharmaceutical composition can be determined, e.g., by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Data obtained from cell culture assays or animal studies can be used in formulating a range of dosage for use in humans. If a pharmaceutical composition is provided systemically, the dosage of such compounds lies preferably within a range of circulating concentrations that include the ED₅₀ with little or no toxicity. The dosage may vary within this range depending upon the dosage form employed and the route of administration utilized. For any compound used in a method described herein, the therapeutically effective dose can be estimated initially from cell culture assays. A dose may be formulated in animal models to achieve a circulating plasma concentration or within the local environment to be treated in a range that includes the IC₅₀ (i.e., the concentration of the test compound that achieves a half-maximal inhibition of mitochondrial fragmentation as determined in cell culture. Such information can be used to more accurately determine useful doses in humans. Levels in plasma may be measured, for example, by liquid chromatography coupled to mass spectrometry.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present.

Moreover, treatment of a subject with a therapeutically effective amount of a pharmaceutical composition described herein can include a single treatment or a series of treatments. For example, several divided doses may be administered daily, one dose, or cyclic administration of the compounds to achieve the desired therapeutic result. A single small molecule compound may be administered, or combinations of various small molecule compounds may also be administered.

It is also possible to add agents that improve the solubility of pharmaceutical compositions. For example, a pharmaceutical composition can be formulated with one or more adjuvants and/or pharmaceutically acceptable carriers according to the selected route of administration. For oral applications, gelatin, flavoring agents, or coating material can be added. In general, for solutions or emulsions, carriers may include aqueous or alcoholic/aqueous solutions, emulsions or suspensions, including saline and buffered media. Parenteral vehicles can include sodium chloride and potassium chloride, among others. In addition, intravenous vehicles can include fluid and nutrient replenishers, electrolyte replenishers and the like.

Numerous coating agents can be used in accordance with various embodiments. In certain embodiments, the coating agent is one which acts as a coating agent in conventional delayed release oral formulations, including polymers for enteric coating. Examples include hypromellose phthalate (hydroxy propyl methyl cellulose phthalate; HPMCP); hydroxypropylcellulose (HPC; such as KLUCEL®); ethylcellulose (such as ETHOCEL®); and methacrylic acid and methyl methacrylate (MAA/MMA; such as EUDRAGIT®).

In certain embodiments, a pharmaceutical composition also includes at least one disintegrating agent, as well as diluent. In some embodiments, a disintegrating agent is a super disintegrant agent. One example of a diluent is a bulking agent such as a polyalcohol. In many embodiments, bulking agents and disintegrants are combined, such as, for example, PEARLITOL FLASH®, which is a ready to use mixture of mannitol and maize starch (mannitol/maize starch). In accordance with a number of embodiments, any polyalcohol bulking agent can be used when coupled with a disintegrant or a super disintegrant agent. Additional disintegrating agents include, but are not limited to, agar, calcium carbonate, maize starch, potato starch, tapioca starch, alginic acid, alginates, certain silicates, and sodium carbonate. Suitable super disintegrating agents include, but are not limited to crospovidone, croscarmellose sodium, AMBERLITE (Rohm and Haas, Philadelphia, Pa.), and sodium starch glycolate.

In certain embodiments, diluents are selected from the group consisting of mannitol powder, spray dried mannitol, microcrystalline cellulose, lactose, dicalcium phosphate, tricalcium phosphate, starch, pregelatinized starch, compressible sugars, silicified microcrystalline cellulose, and calcium carbonate.

In certain embodiments, a pharmaceutical composition further utilizes other components and excipients. For example, sweeteners, flavors, buffering agents, and flavor enhancers to make the dosage form more palatable. Sweeteners include, but are not limited to, fructose, sucrose, glucose, maltose, mannose, galactose, lactose, sucralose, saccharin, aspartame, acesulfame K, and neotame. Common flavoring agents and flavor enhancers that may be included in the formulations described herein include, but are not limited to, maltol, vanillin, ethyl vanillin, menthol, citric acid, fumaric acid, ethyl maltol and tartaric acid.

In certain embodiments, a pharmaceutical composition also includes a surfactant. In certain embodiments, surfactants are selected from the group consisting of Tween 80, sodium lauryl sulfate, and docusate sodium.

In certain embodiments, a pharmaceutical composition further utilizes a binder. In certain embodiments, binders are selected from the group consisting of povidone (PVP) K29/32, hydroxypropylcellulose (HPC), hydroxypropylmethylcellulose (HPMC), ethylcellulose (EC), corn starch, pregelatinized starch, gelatin, and sugar.

In certain embodiments, a pharmaceutical composition also includes a lubricant. In certain embodiments, lubricants are selected from the group consisting of magnesium stearate, stearic acid, sodium stearyl fumarate, calcium stearate, hydrogenated vegetable oil, mineral oil, polyethylene glycol, polyethylene glycol 4000-6000, talc, and glyceryl behenate.

Preservatives and other additives, like antimicrobial, antioxidant, chelating agents, and inert gases, can also be present. (See generally, Remington's Pharmaceutical Sciences, 16th Edition, Mack, (1980), the disclosure of which is incorporated herein by reference.)

### Modes of Treatments

In certain embodiments, compounds are administered in a therapeutically effective amount as part of a course of treatment of a metabolic disorder. As used in this context, to “treat” means to ameliorate or prevent at least one symptom of a metabolic disorder to be treated or to provide a beneficial physiological effect. For example, amelioration of a symptom could be improvement of insulin sensitivity, improvement of glucose tolerance, improvement of leptin sensitivity, a reduction in leptin levels, and increase in adiponectin levels, and decrease in hepatic steatosis, and/or reduction of body fat.

A number of embodiments are directed towards treating an individual for a metabolic disorder. Accordingly, an embodiment to treat an individual is as follows:
(i) diagnose or determine that an individual has a metabolic disorder
(ii) administer to the individual a sphingolipid-like compound, an ARF6 antagonist, and/or a PIKfyve antagonist

In certain embodiments, an individual to be treated has been diagnosed as having a metabolic disorder. Metabolic disorders include (but are not limited to) obesity, metabolic syndrome, hyperglycemia, type 2 diabetes, insulin resistance, leptin resistance, hyperleptinemia, and hepatic steatosis (e.g., nonalcoholic hepatic steatosis (NASH)).

A therapeutically effective amount can be an amount sufficient to prevent reduce, ameliorate or eliminate the symptoms of diseases or pathological conditions susceptible to such treatment, such as, for example, insulin insensitivity, glucose intolerance, leptin insensitivity, hyperleptinemia, low plasma adiponectin levels, hepatic steatosis, and/or obesity. In certain embodiments, a therapeutically effective amount is an amount sufficient to antagonize mitochondrial fragmentation.

### Methods of Mitigating Mitochondrial Fragmentation

In certain embodiments, a biological cell is contacted with a compound to mitigate, prevent, and/or reverse mitochondrial fragmentation. In certain embodiments, a biological to be contacted is a cell experiencing fragmentation of mitochondria. In certain embodiments, a biological cell is associated with a metabolic disorder, including (but not limited to) obesity, metabolic syndrome, hyperglycemia, type 2 diabetes, insulin resistance, leptin resistance, hyperleptinemia, and hepatic steatosis (e.g., nonalcoholic hepatic steatosis (NASH)).

A number of embodiments are directed towards treating a biological cell for mitigating, preventing, and/or reversing mitochondrial fragmentation Accordingly, an embodiment to treat a biological cell is as follows:
(i) provide a biological cell experiencing fragmentation of mitochondria
(ii) contact the biological cell with a sphingolipid-like compound, an ARF6 antagonist, and/or a PIKfyve antagonist

In certain embodiments, a compound for treatment of a biological cell is utilized at concentration between 1 nM to 100 μM. In certain embodiments a compound is utilized at concentration on the order of less than 1 nM, 1 nM, 10 nM, 100 nM, 1 μM, 10 μM, 100 μM, or greater than 100 μM.

### EXEMPLARY EMBODIMENTS

Biological data supports the use of the aforementioned sphingolipid-like compounds in a variety of embodiments to treat metabolic disease. The therapeutic efficacy of sphingolipid-like small molecule embodiments stems from its demonstrated biological activity in preliminary studies in human and mouse cells and mouse models of metabolic disorders.

### Example 1: A Drug-Like Sphingolipid Corrects Obesity by Reversing Ceramide-Induced Mitochondrial Fragmentation

Obesity has emerged as a serious epidemic. According to the World Health Organization, an estimated 13% of the world's adult population was obese in 2016, a number that has nearly tripled since 1975. Even more alarming is the accelerating prevalence of obesity in children. Worldwide, over 340 million children aged 5-19 were overweight or obese in 2016; most of these children will eventually become obese adults. Because obesity and related comorbidities are leading causes of preventable and pre-mature death, these statistics reflect a staggering social and economic burden. While the drivers of the growing obesity epidemic are multi-factorial, over-consumption of calorie dense, high-fat foods clearly contributes. These hypercaloric diets synergize with environmental and genetic factors to create a chronic positive energy balance that leads to excessive adiposity. While dietary modification and increasing exercise are an integral part of any interventional program, lifestyle changes have proven insufficient to resolve obesity in most patients. The “eat less and move more” approach ignores the complex physiologic, psychological, and genetic factors that prevent some patients from maintaining a negative energy balance. While bariatric surgery is highly effective in many patients, it is also invasive and can be accompanied by serious complications. There is thus a critical unmet need for medical therapies that can complement lifestyle interventions and help individuals overcome the barriers to successful long-term weight loss.

FDA-approved weight-loss agents are only marginally effective and often plagued by toxicities and side effects. Increased understanding of the signals that control satiety and metabolism, especially the hormonal crosstalk between peripheral tissues and complex neural circuitry, has identified several new targets that may be more amenable to pharmacological intervention. The discovery of the hormone leptin was particularly exciting, offering hope that obesity could be treated by modulating leptin signaling. Although pharmacological administration of leptin dramatically decreases food intake and increases energy expenditure in the rare patients with leptin deficiency, leptin itself has limited potential as an obesity therapeutic. The majority of obese individuals are hyperleptinemic and resistant to the effects of exogenous leptin, a phenotype that has been traced to disruptions in the balance of mitochondrial fission and fusion that lead to a fragmented mitochondrial network (C. A. Galloway & Y. Yoon, Antioxid. Redox Signal. 19: 415-430, 2013; E. Schrepfer & L. Scorrano, Mol. Cell 61: 683-694, 2016; and T. Wai & T. Langer, Trends Endocrinol. Metab. 27: 105-117, 2016; the disclosures of which are incorporated herein by reference). Agents that overcome leptin resistance remain an aspirational goal. Restoring leptin sensitivity by reversing mitochondrial fragmentation is a particularly appealing strategy as insulin resistance and hepatic steatosis also follow from excessive mitochondrial fission (C. A. Galloway, et al., Am. J. Physiol. Gastrointest. Liver Physiol. 307: G632-41, 2014; B. M. Filippi, et al., Cell Rep. 18: 2301-2309, 2017; H-F. Jheng, et al., Mol. Cell. Biol. 32: 309-319, 2012; L. Wang, et al., Diabetologia 58: 2371-2380, 2015; D. Sebastien, et al., Proc. Natl. Acad. Sci. USA 109: 5523-5528, 2012; the disclosures of which are incorporated herein by reference). Reducing mitochondrial fragmentation is expected to lower plasma leptin levels which, somewhat paradoxically, has been demonstrated to improve leptin sensitivity and protect from diet-induced obesity (S. Zhao, et al., Cell Metab. 30:706-719, 2019; and G. Mancini, et al., 26:2849-2858, 2019; the disclosures of which is incorporated herein by reference). In summary, the poorly stocked armamentarium and the expanding scope of the obesity epidemic provides a strong impetus to develop and test innovative therapeutic approaches, particularly agents that limit mitochondrial fission.

Diet-induced obesity causes mitochondrial fragmentation downstream of increased C16:0 ceramide production (S. Choi & A. J. Snider, Mediators Inflamm. 2015: 520618, 2015; J. A. Chavez & S. A. Summers, Cell Metab. 15: 585-594, 2012; S. A. Summers, B. Chaurasia & W. L. Holland, Nat. Metab. 1: 1051-1058, 2019; P. Hammerschmidt, et al., Cell 177: 1536-1552, 2019; the disclosures of which are incorporated herein by reference). The Western diet is high in saturated fat which leads to increased circulating levels of palmitate, supplying both the backbone and fatty acid chain for C16:0 ceramide synthesis. Reducing ceramide production by deleting serine palmitoyl transferase (SPT), ceramide synthase 6 (CerS6), or dihydroceramide desaturase 1 (DES1) protects mice from the negative metabolic consequences of consuming a HFD in part by preventing mitochondrial fragmentation (Z. Li, et al., Mol. Cell. Biol. 31: 4205-4218, 2011; S. M. Turpin, et al., Cell Metab. 20: 678-686, 2014; W. L. Holland, et al., Cell Metab. 5: 167-179, 2007; and B. Chaurasia, et al., Science 365: 386-392, 2019; the disclosures of which are incorporated herein by reference; see also P. Hammerschmidt, et al., 2019, cited supra). Unfortunately, drugs that safely and selectively target these enzymes are not yet available. The small molecule SPT inhibitor myriocin is effective in mice but is not a viable therapeutic. Global inhibitors of all six ceramide synthase isoforms are poor clinical candidates given the diverse structural and signaling roles played by various ceramide isoforms. A selective CerS6 inhibitor should be better tolerated and would correct mitochondrial fragmentation and metabolic dysfunction in HFD-fed mice (S. Raichur, et al., Mol. Metab. 21: 36-50, 2019, the disclosure of which is incorporated herein by reference; see also Hammerschmidt, et al., 2019 and S. M. Turpin, 2014, cited supra). Because the water-soluble, orally-bioavailable synthetic sphingolipid sphingolipid-like compound 893 is structurally related to established CerS inhibitors, its ability to prevent palmitate-induced C16:0 ceramide production and mitochondrial fission was evaluated. sphingolipid-like compound 893 did not inhibit CerS6 as it failed to block C16:0 ceramide generation (J. Chen, et al., J. Endocrinol. 237: 43-58, 2019; and N. Turner, et al., Nat. Commun. 9: 3165, 2018; the disclosures of which are incorporated herein by reference). However, it did provide robust protection from both ceramide- and palmitate-induced mitochondrial fragmentation and was therefore evaluated as an interventional agent in HFD-fed mice.

Sphingolipid-Like Compound 893 Protects from Ceramide-Induced Mitochondrial Fragmentation.

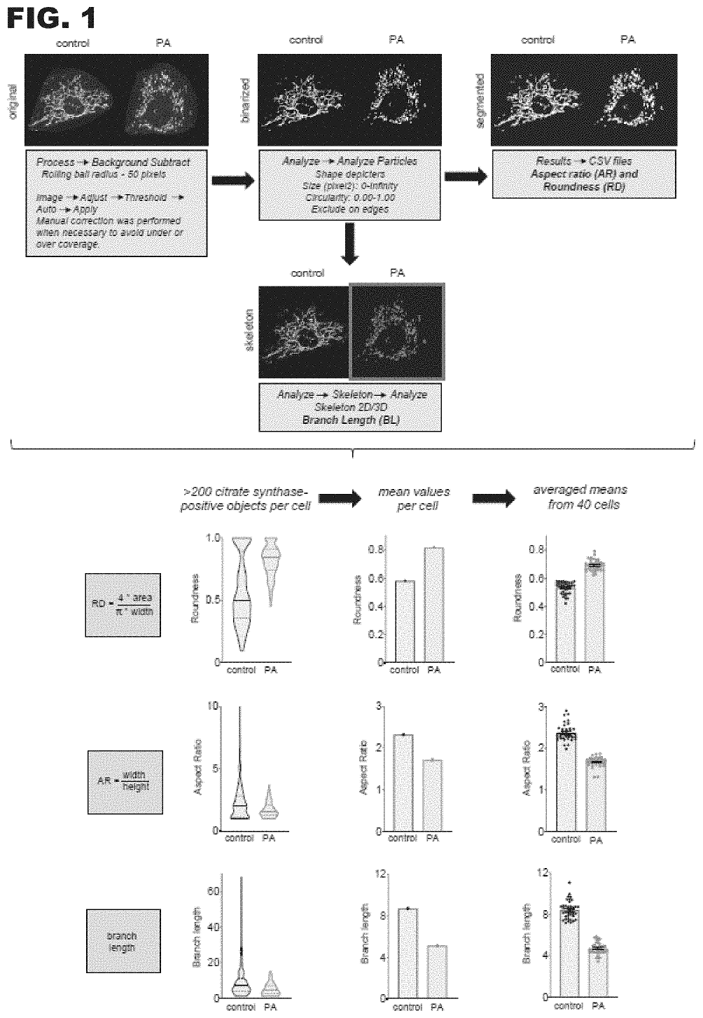

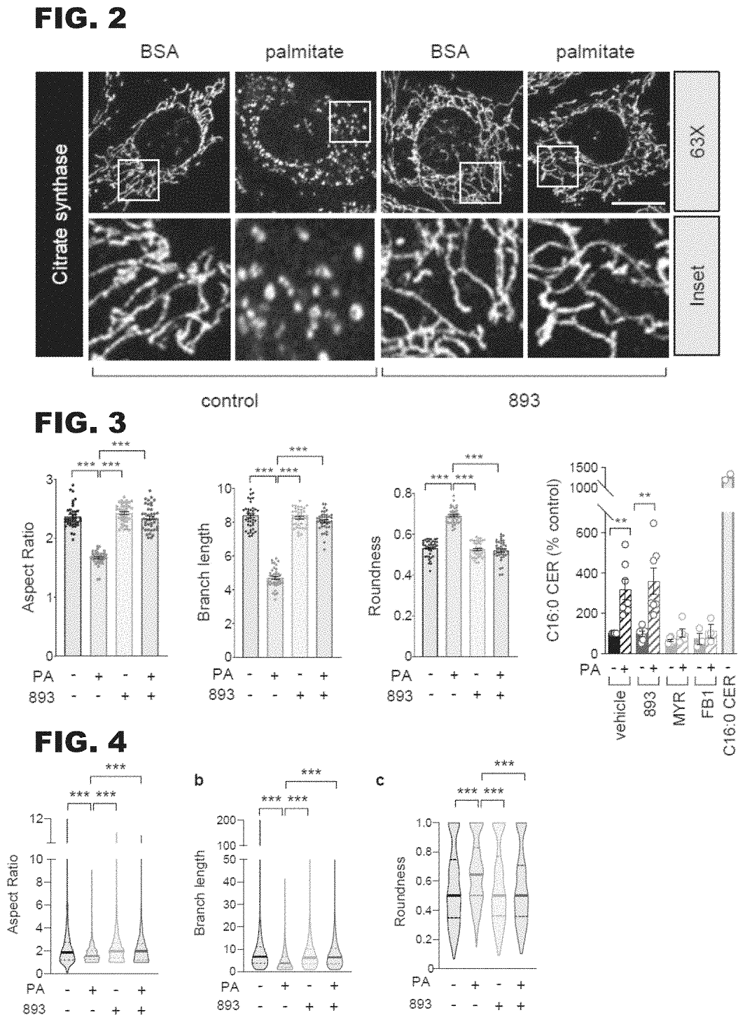

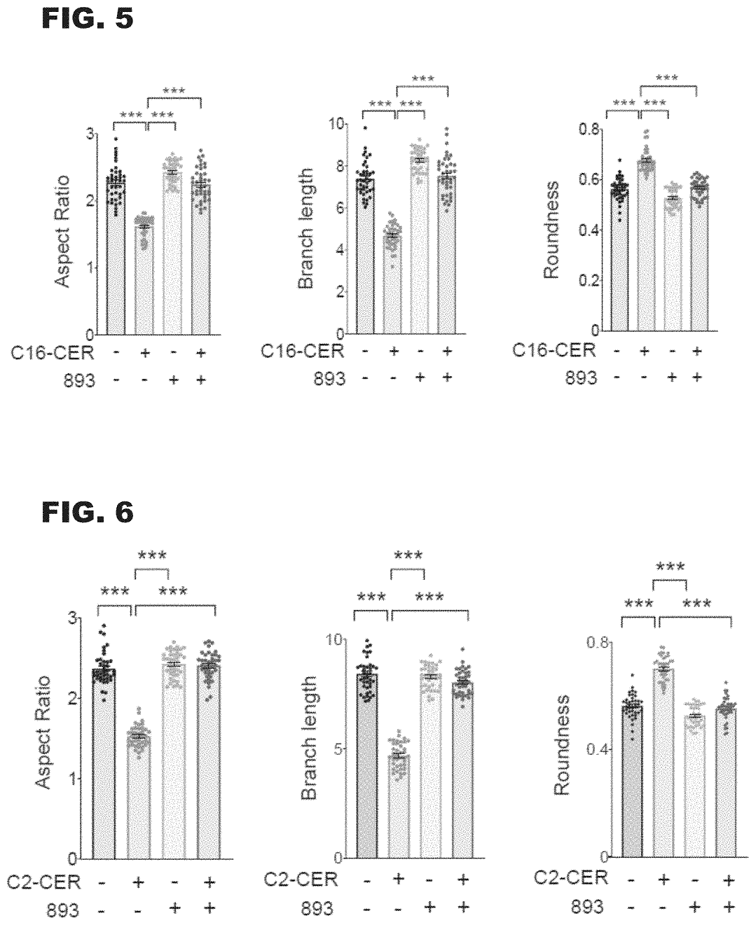

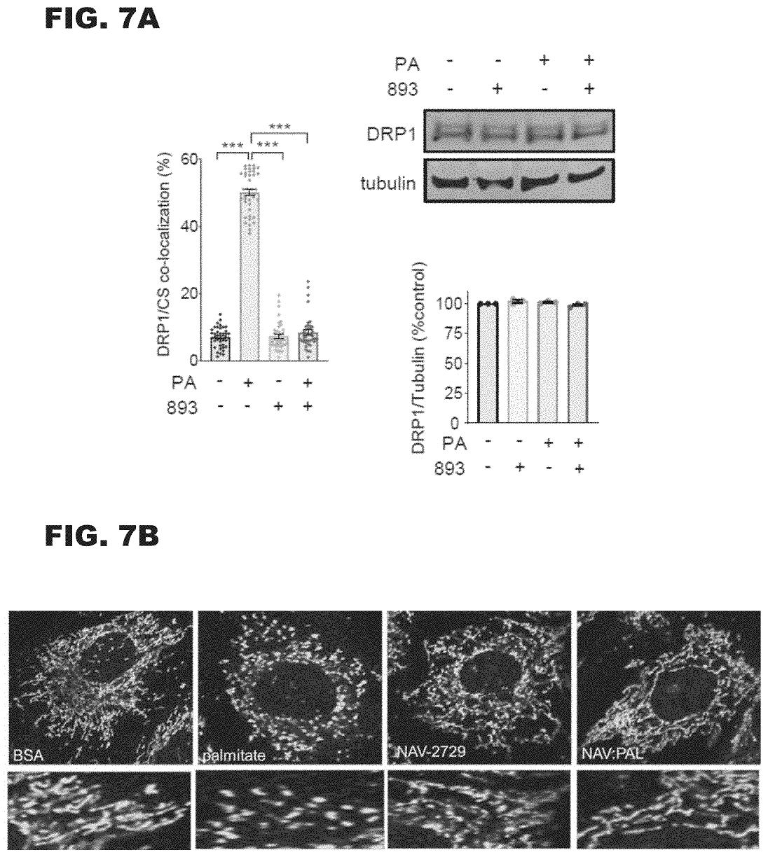

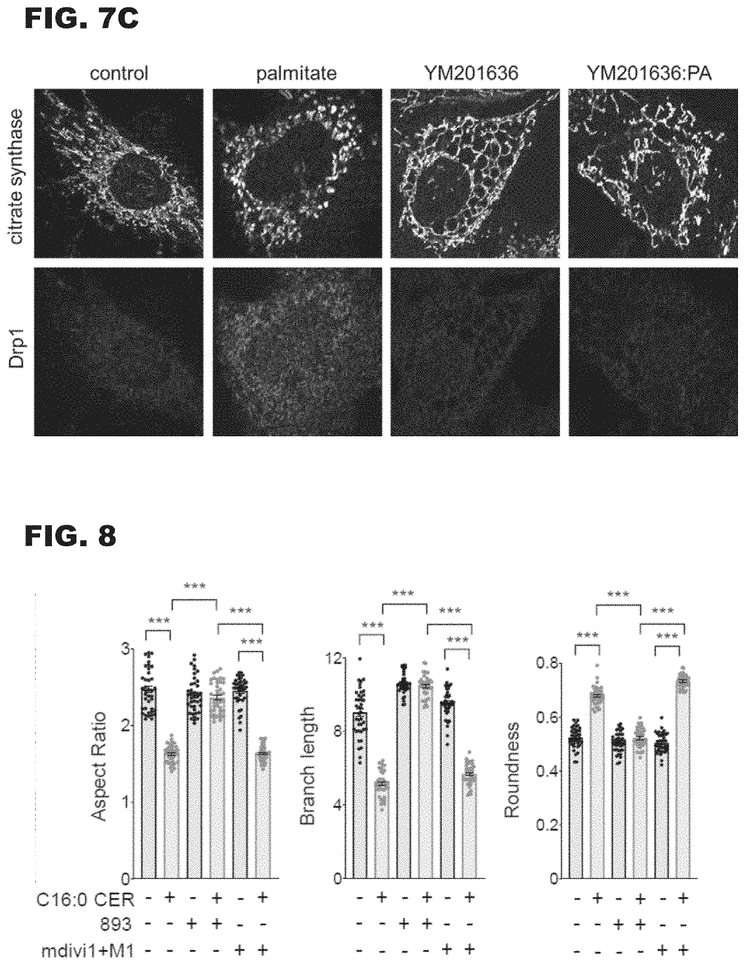

Mice consuming a HFD experience increases in circulating palmitate that is converted to C16:0 ceramide, triggering the mitochondrial fragmentation that is responsible for many of the negative metabolic consequences of obesity (M. Schneeberger, et al., Cell 155: 172-187, 2013; and M. E. Smith, et al., Biochem. J. 456: 427-439; the disclosures of which are incorporated herein by reference; see also, H-F. Jheng, et al., 2012; B. M. Filippi, et al., 20107; Hammerschmidt, et al., 2019; L. Wang, et al., 2015; and D. Sebastien, et al., 2012; cited supra). The structural similarity between the synthetic sphingolipid sphingolipid-like compound 893 and compounds that inhibit ceramide synthases prompted an evaluation of whether sphingolipid-like compound 893 limits ceramide generation and mitochondrial fission in palmitate-exposed cells. Murine embryonic fibroblasts (MEFs) possess a highly tubular mitochondrial network simplifying the detection of increased mitochondrial fission. Mitochondrial morphology was assessed by examining citrate synthesis expression under a high resolution microscopy. Specifically, the aspect ratio, branch length and roundness of mitochondria was assessed (FIG. 1). Palmitate supplementation increased C16:0 ceramide levels and produced dramatic mitochondrial fragmentation in MEFs as expected (FIGS. 2 to 4). Blocking ceramide production with either the SPT inhibitor myriocin or the general ceramide synthase inhibitor fumonisin B1 prevented palmitate-induced morphological changes, maintaining mitochondrial tubule length (aspect ratio and branch length) and preventing the increase in mitochondrial roundness. Like these inhibitors of ceramide synthesis, sphingolipid-like compound 893 preserved a tubular, branched mitochondrial network in palmitate-treated cells (FIGS. 2 to 4). However, sphingolipid-like compound 893 maintained mitochondrial morphology without blocking palmitate-induced ceramide production (FIG. 3). Indeed, the effects of sphingolipid-like compound 893 on mitochondrial dynamics lie downstream of ceramide generation as sphingolipid-like compound 893 also blocked ceramide-induced mitochondrial fragmentation (FIGS. 5 & 6). Mechanistically, sphingolipid-like compound 893 prevented the recruitment of the GTPase that mediates fission, DRP1, to mitochondrial membranes in response to palmitate without affecting DRP1 protein expression levels (FIG. 7A). Sphingolipid-like compound 893 inactivates ARF6 and PIKfvye. Consistent with this, NAV-2729, an inhibitor of ARF6, and YM201636, an inhibitor of PIKfyve, offered partial protection from palmitate-induced mitochondrial fragmentation (FIGS. 7B & 7C). Further, YM201636 prevented DRP1 recruitment. Thus, sphingolipid-like compound 893 maintains a tubulated mitochondrial network by blocking DRP1 recruitment, most likely downstream of ARF6 and PIKfyve inactivation. In sum, the synthetic sphingolipid-like compound 893 prevents palmitate-induced mitochondrial fragmentation downstream of ceramide synthesis likely by inactivating ARF6 and PIKfyve, blocking ceramide-induced recruitment of DRP1 to mitochondria.

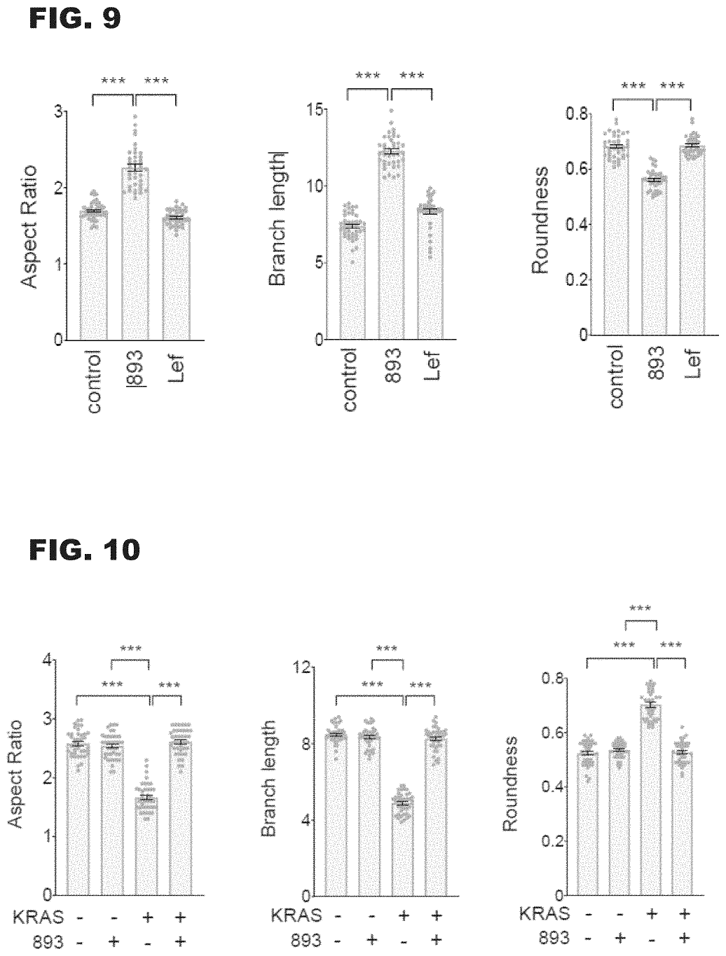

Genetic studies conducted in mice suggest that small molecules that prevent mitochondrial fragmentation could have significant therapeutic value in obese patients (A. Santoro, et al., Cell Metab. 25: 647-660, 2017, the disclosure of which is incorporated herein by reference; see also L. Wang, et al., 2015; D. Sebastien, et al., 2012; and M. Schneeberger, et al., 2013; cited supra). Mdivi-1 has been widely employed as an inhibitor of the DRP1 GTPase and has been evaluated in obesity models. Mdivi-1 reduces ROS production in palmitate- or ceramide-treated C2C12 myotubes, moderately improves insulin resistance without affecting glucose clearance in ob/ob mice, restores insulin-mediated suppression of hepatic glucose production in HFD-fed rats, and slows the progression of diabetic nephropathy in db/db mice. However, compelling evidence suggests that the limited benefits of mdivi-1 in obesity models stem from mitochondrial complex I inhibition, not DRP1 inactivation. Indeed, mdivi-1 from two different suppliers failed to prevent C16:0 ceramide-induced mitochondrial fragmentation even after a prolonged pre-incubation or when combined with the small molecule mitochondrial fusion promoter M1 (FIG. 8). While celastrol sensitizes to leptin and protects from HFD-induced obesity, it did not protect from palmitate-induced mitochondrial fission, instead triggering severe mitochondrial fragmentation as a single agent. The cell-permeant peptide inhibitor, P110, blocks DRP1 from interacting with FIS1 on the outer mitochondrial membrane. While sphingolipid-like compound 893 was effective after only 1 h, a prolonged pre-treatment was necessary for P110 to prevent C16:0 ceramide-induced mitochondrial fragmentation. The rheumatoid arthritis therapeutic leflunomide is the only FDA-approved drug shown to promote mitochondrial fusion. Consistent with its mechanism of action, transcriptional induction of the mitochondrial fusion factors MFN1 and MFN2, leflunomide blocked C16:0 ceramide-induced mitochondrial fragmentation after a 24 h, but not a 1 h, pre-incubation. Moreover, although leflunomide has been reported to promote mitochondria fusion in KRAS-mutant cancer cells (M. Yu, et al., JCI Insight. 5:e126915, 2019, the disclosures of which is incorporated herein by reference), its effects are context-dependent as it did not reverse mitochondrial fragmentation in A549 lung cancer cells expressing oncogenic KRAS^{G12D} (FIG. 9). In contrast, sphingolipid-like compound 893 rapidly corrected KRAS-mediated mitochondrial fission in both A549 lung cancer cells and KRAS G12D knock-in MEFs (FIG. 10). Further, the ceramide synthase inhibitors myriocin or fumonisin B1 did not increase mitochondrial tubularity in A549 cells, demonstrating that sphingolipid-like compound 893 opposes mitochondrial fission in response to signals other than ceramide. Together, these experiments show that sphingolipid-like compound 893 is more effective, more potent, and/or acts more rapidly than compounds previously reported to modulate mitochondrial dynamics.

Sphingolipid-Like Compound 893 Protects from HFD-Induced Mitochondrial Fragmentation.

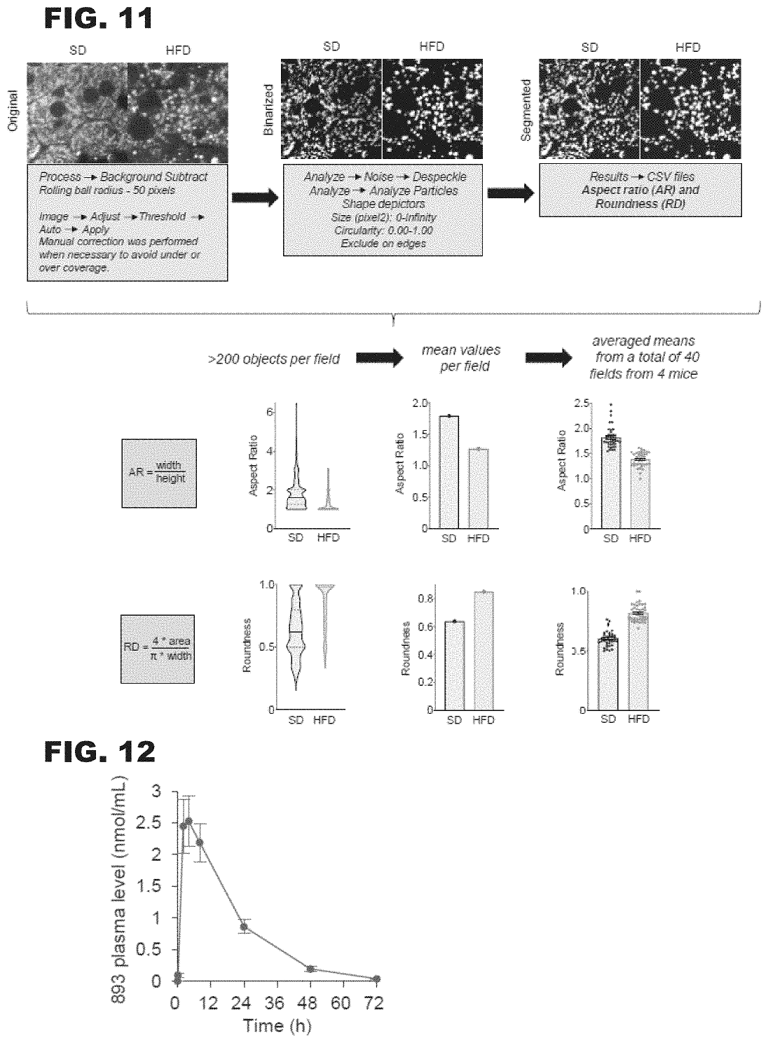

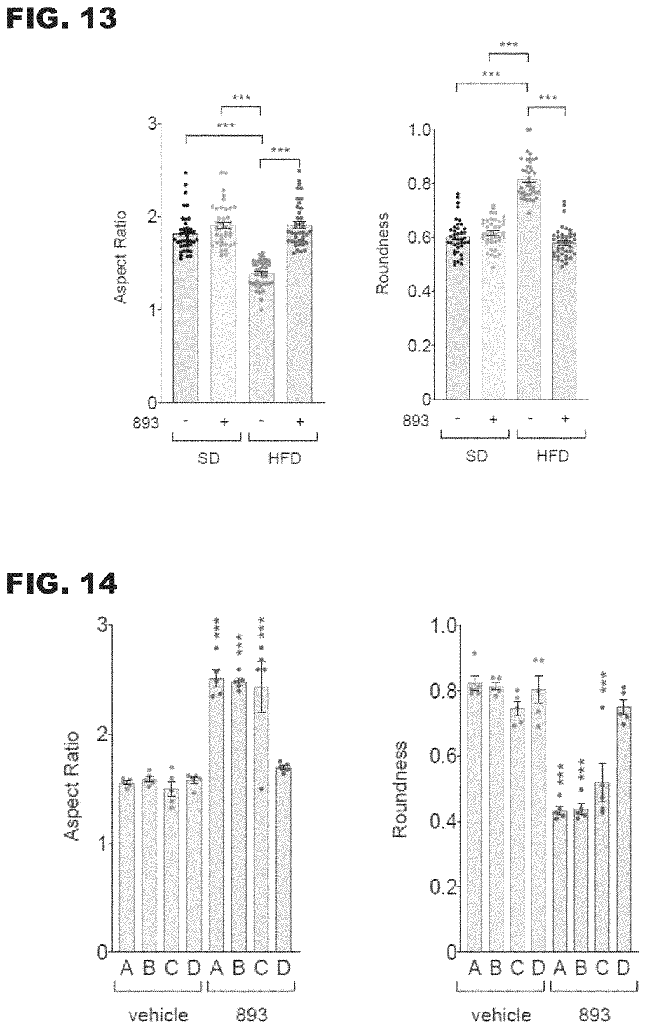

To determine whether sphingolipid-like compound 893 also protects from ceramide-induced mitochondrial fragmentation in vivo, a cohort of mice with diet-induced obesity was analyzed. Male, C57BL/6J mice were fed a 45% kcal from fat rodent diet (HFD) or a standard chow diet (10% kcal from fat) for 22-26 weeks. Mitochondrial morphology was compared in freshly resected livers from vehicle- or sphingolipid-like compound 893-treated mice using NADH/NADPH autofluorescence and confocal microscopy. When evaluating mitochondrial morphology, light microscopy has two, significant advantages over electron microscopy: 1) the 3D architecture of the mitochondrial network is readily apparent, and 2) quantitative measurements of mitochondrial shape can be made in a large number of cells in an automated and unbiased manner using image analysis software (FIG. 11). In addition, evaluating morphology of intact, viable mitochondria avoids artifacts introduced by tissue processing or lengthy hepatocyte isolation procedures. Because the morphology of liver mitochondria varies over the circadian cycle, vehicle- and sphingolipid-like compound 893-treated mice were sacrificed in pairs between ZT13 and ZT17, a time frame when mitochondria were expected to be maximally fragmented in HFD-fed mice. Based on the pharmacokinetics of sphingolipid-like compound 893 (tₘₐₓ=4 h, t_{1/2}=10.6 h, FIG. 12), animals were treated at ZT8.5, 3.5 h before the onset of the dark period. sphingolipid-like compound 893 was administered at 120 mg/kg by oral gavage based on prior studies demonstrating that this dose inhibits tumor growth and reduces amino acid-dependent mTORC1 signaling without toxicity as assessed by blood chemistry, complete blood count, and liver and small intestine histology (S. M. Kim, et al., J. Clin. Invest. 126: 4088-4102, 2016, the disclosure of which is incorporated herein by reference). Mitochondria in the livers of mice chronically maintained on a HFD were larger and more spherical than those in the livers of mice fed the SD (FIG. 13). Administration of a single oral dose of 120 mg/kg sphingolipid-like compound 893 to treatment-naïve, HFD-fed mice at ZT8.5 caused a dramatic change in the morphology of hepatic mitochondria, increasing their tubularity (increased aspect ratio) and reducing their roundness to match controls fed standard chow. Sphingolipid-like compound 893 did not significantly alter hepatic mitochondrial morphology in lean mice consuming the SD. Thus, sphingolipid-like compound 893 acutely corrects aberrant mitochondrial morphology in the livers of obese, HFD-fed mice.

Preventing mitochondrial fission in the liver improves insulin sensitivity in mice with diet-induced obesity, while blocking fission in anorexigenic hypothalamic POMC neurons reduces food intake by restoring leptin sensitivity (A. Santoro, et al., 2017; and M. Schneeberger, et al., 2013, cited supra). To determine whether sphingolipid-like compound 893 was also effective in the hypothalamus, mitochondrial morphology was assessed in the brains of the same mice evaluated in FIG. 13. Due to the increased time required to remove the organ, brain mitochondria were visualized in fixed tissues by immunofluorescence microscopy rather than by NADH/NADPH autofluorescence. Consistent with the abnormal mitochondrial morphology in the livers of the same HFD-fed mice, citrate synthase staining revealed round, swollen mitochondria in the hypothalamus and cerebral cortex (FIG. 14). Sphingolipid-like compound 893 reversed these HFD-induced changes in mitochondrial morphology in the brains of 3 out of 4 animals examined (FIG. 14). The sphingolipid-like compound 893-treated animal with fragmented brain mitochondria was the last to be evaluated suggesting that sphingolipid-like compound 893 levels in the brain may be lower and/or fall more quickly than in the liver where results were homogeneous (FIGS. 13 & 14). In sum, orally administered sphingolipid-like compound 893 acutely reversed pathological, HFD-induced changes in mitochondrial morphology in multiple tissues that play critical roles maintaining metabolic homeostasis.

The protein Mfn2 plays a central role in mediating mitochondrial fusion. Genetic deletion of this protein from various tissues results in profound mitochondrial fragmentation that mimics that observed in diet-induced obesity (G. Mancini, et al., 2019, cited supra). In mouse models, the loss of Mfn2 from mature adipocytes is sufficient to produce obesity. This mitochondrial fragmentation in adipocytes leads to significantly elevated leptin production and reductions in plasma adiponectin resulting in increased food intake. Intriguingly, reducing but not eliminating leptin levels in obese mice was recently shown to improve the response to leptin by removing feedback inhibition of leptin signaling (S. Zhao, et al., 2019, cited supra). The potential therapeutic value of leptin reduction in treating obesity was demonstrated using neutralizing antibodies to leptin in murine diet-induced obesity models. Consistent with its ability to reduce mitochondrial fragmentation in multiple tissues in vivo, 893 reduced leptin production by adipocytes in vivo. Thus, 893 represents a strategy to therapeutically lower leptin levels in obese patients in order to restore sensitivity to this anti-obesity hormone.

### Sphingolipid-Like Compound 893 Restores Normal Body Weight and Adiposity in Mice Consuming a HFD.

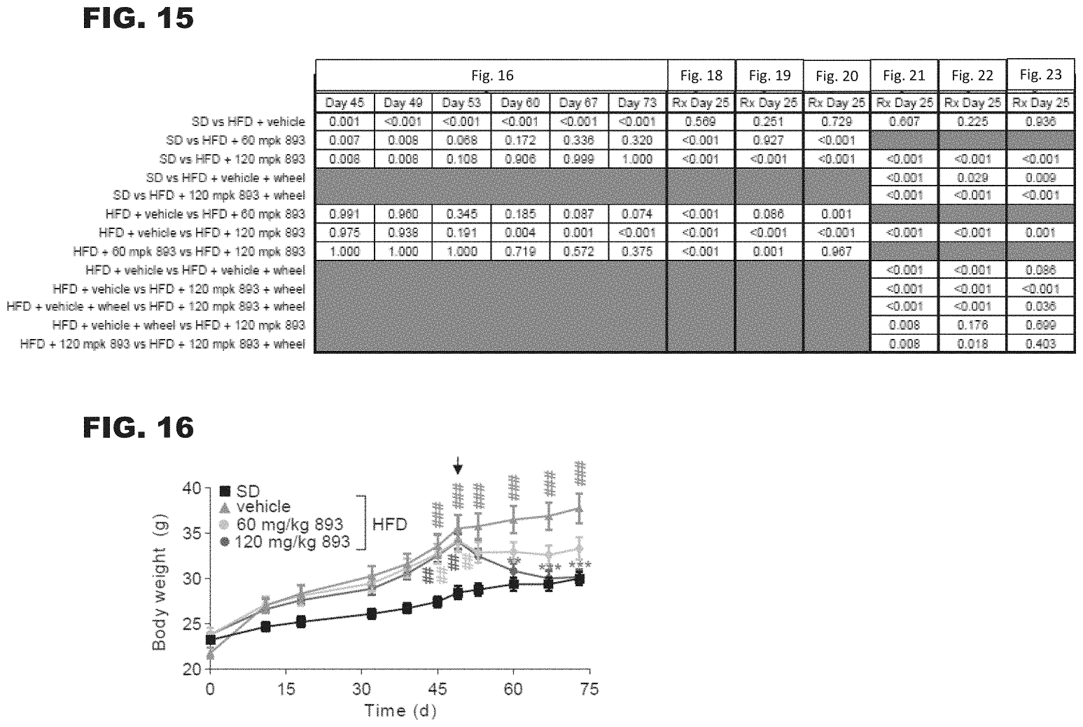

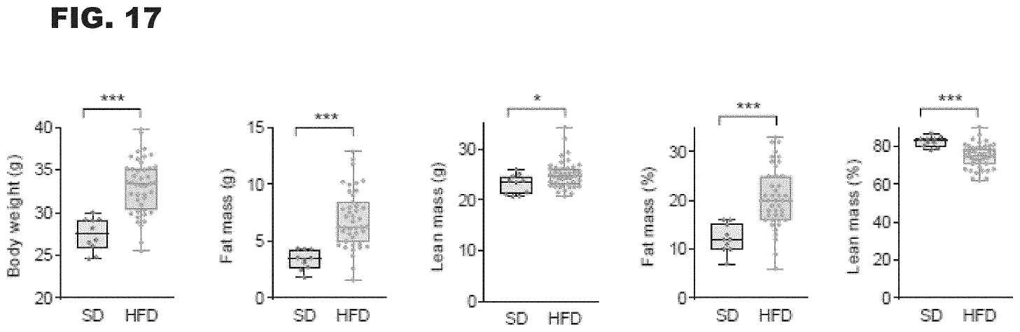

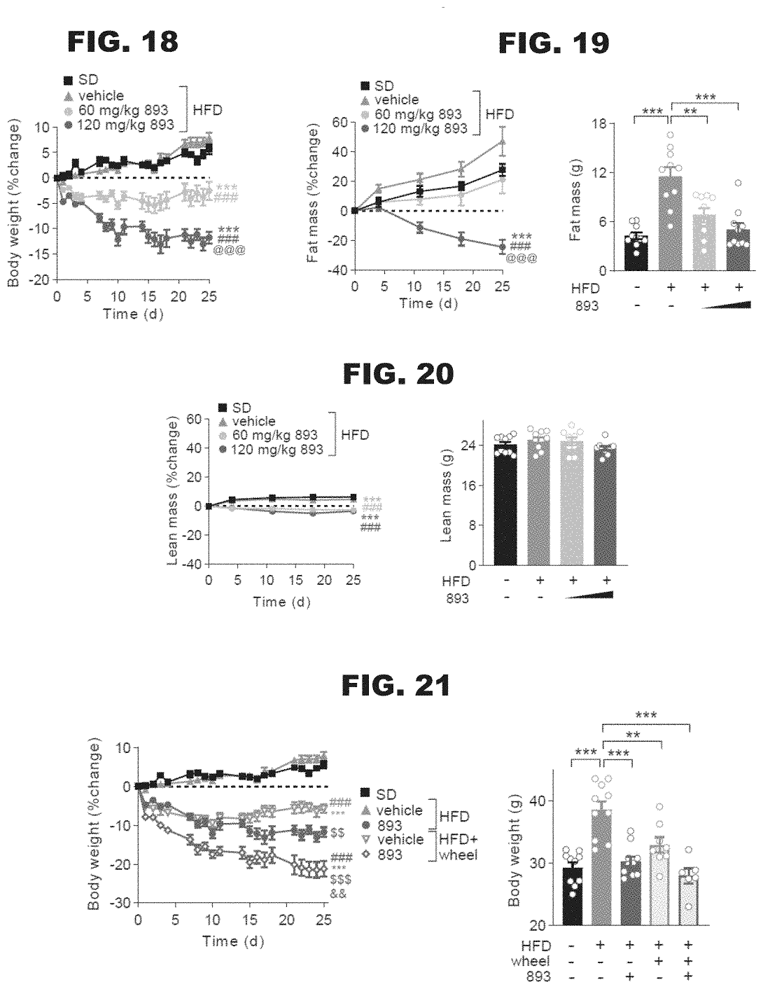

Given its ability to correct HFD-induced mitochondrial fragmentation in vivo, sphingolipid-like compound 893 was evaluated as an interventional therapy for diet-induced obesity. Six-week old male, C57BL/6J mice were fed the HFD for 45 days; an age-matched cohort of control mice was maintained on the SD throughout the study. After 45 days, the average body weight of HFD-fed mice was approximately 130% that of chow-fed mice (FIGS. 15 to 17). Using quantitative nuclear magnetic resonance imaging, fat mass represented 21% of the body weight of mice that had consumed the HFD for 45 days and 12% of body weight in controls fed the standard diet; changes in lean body mass were of lesser magnitude (FIG. 17). At this point, HFD-fed mice were randomly assigned to receive vehicle (water), 60 mg/kg, or 120 mg/kg sphingolipid-like compound 893 by gavage; SD mice were treated with vehicle. Based on the increased activity of mice during the dark cycle (ZT12-ZT24) and the plasma pharmacokinetics of orally administered sphingolipid-like compound 893 (see FIG. 12), mice were treated at ZT8.5 on Mondays, Wednesdays, and Fridays. While the vehicle-treated HFD group continued to gain weight as expected, mice treated with 60 mg/kg or 120 mg/kg sphingolipid-like compound 893 exhibited dose-dependent weight loss despite continued consumption of the HFD (FIG. 16). In the group receiving 120 mg/kg sphingolipid-like compound 893, the rate of weight loss slowed after 10 days. After 2 weeks (6 doses of sphingolipid-like compound 893), the body weight of mice eating the HFD and treated with 120 mg/kg sphingolipid-like compound 893 was no longer statistically different from that of mice continuously fed a standard chow diet (FIGS. 15 and 16); the 60 mg/kg group no longer gained weight, but did not match chow-fed controls. Despite continued treatment with the high dose of sphingolipid-like compound 893, weight loss plateaued once body weight matched that of SD controls (FIGS. 16 and 18). The majority of the dose-dependent weight loss in sphingolipid-like compound 893-treated mice was due to a decline in fat mass with little change in lean mass indicating that overall body composition was improved (FIGS. 15, 19 & 20). Mice treated with 60 mg/kg sphingolipid-like compound 893 gained fat mass at a similar rate to mice fed a standard diet (FIG. 19) indicating that this dose was sufficient to prevent adiposity resulting from HFD feeding. As in a prior report where mice were dosed 5-7 days a week for 11 weeks (S. M. Kim, et al., 2016, cited supra), sphingolipid-like compound 893 was well-tolerated, and the behavior of sphingolipid-like compound 893-treated mice was overtly normal throughout the study. These results indicate that sphingolipid-like compound 893 restores normal adiposity and body weight in previously obese mice despite the continuous feeding of a HFD.

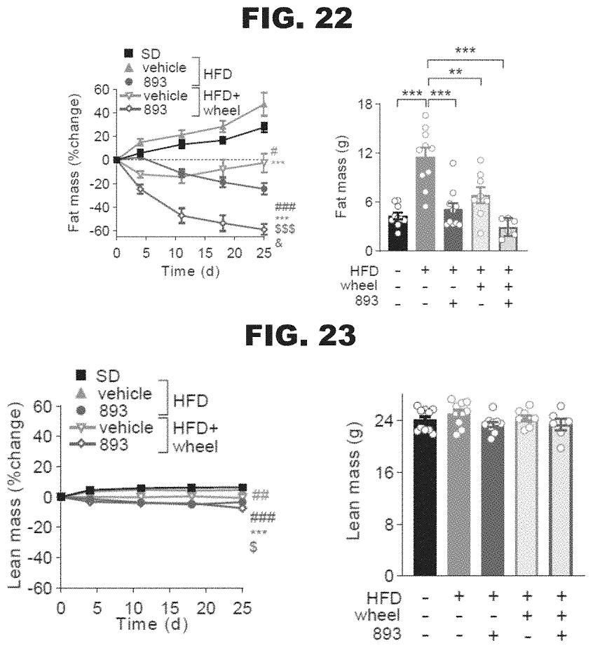

Exercise can mitigate the negative effects of hyper-nutrition. When provided with a running wheel, mice will voluntarily run 2-10 km per night, slowing the body weight and fat gain that normally accompany HFD feeding and improving metabolic status. To benchmark the effects of sphingolipid-like compound 893 against voluntary exercise and to determine whether the beneficial effects of these interventions are additive, 16 male, C57BL/6J mice that had been fed a HFD for 7 weeks were individually housed, provided with running wheels, and randomly assigned to receive vehicle or 120 mg/kg sphingolipid-like compound 893 on the Monday/Wednesday/Friday schedule. Rodent running activity declines under stress, and monitoring the duration and distance of voluntary wheel running also provides a holistic measure of overall mouse health. HFD-fed mice receiving vehicle ran an average daily distance of 2.8±0.7 km over the course of the experiment, a value that was not significantly different from the sphingolipid-like compound 893-treated group (2.8±1.2 km). The average time spent on running wheels each day was also equivalent in vehicle- and sphingolipid-like compound 893-treated groups. Exercise activity was generally well-matched between the groups on a given day suggesting that day to day differences in activity were likely related to uncontrolled variations in the environment. As expected, voluntary exercise led to weight loss in vehicle-treated mice maintained on a HFD that leveled off after the first week of intervention (FIGS. 15, 21, & 22). HFD-fed mice receiving vehicle and housed with a running wheel exhibited a similar body weight loss to sphingolipid-like compound 893-treated mice maintained in normal caging. Mice both provided with a running wheel and treated with sphingolipid-like compound 893 exhibited even greater weight loss than observed with either treatment alone. Wheel running reduced fat mass while maintaining lean mass in all groups (FIGS. 15, 22 & 23). Together, these results demonstrate that sphingolipid-like compound 893 reduces adiposity and body weight to an equivalent extent as and additively with voluntary exercise and confirm that the effects of sphingolipid-like compound 893 on body weight are unrelated to morbidity or malaise.

Sphingolipid-Like Compound 893 Corrects Metabolic Defects Associated with HFD Feeding.

Chronic over-nutrition leads to toxic lipid accumulation (lipotoxicity) in muscle and liver. Excessive hepatic lipid accumulation can eventually lead to liver fibrosis and inflammation (non-alcoholic steatohepatitis) and cancer. As expected, the livers of vehicle-treated mice maintained on the HFD accumulated excess. Strikingly, treatment with 120 mg/kg sphingolipid-like compound 893 eliminated hepatic steatosis in HFD-fed mice. Unbiased lipidomic analysis revealed that the majority of the lipids that accumulated in the liver on the HFD were triacylglycerols. Ceramides, particularly C16:0 ceramide in the liver and C18:0 ceramide in muscle, also increase with HFD feeding and contribute to the insulin resistance that accompanies diet-induced obesity (S. M. Turpin-Nolan, et al., Cell Rep. 26: 1-10.e7, 2019; N. Turner, et al., Diabetologia 56: 1638-1648, 2013; and M. K. Montgomery, et al., Biochim. Biophys. Acta 1861: 1828-1839, 2016; the disclosures of which are incorporated herein by reference; see also S. M. Turpin, et al., 2014; N. Turner, et al., 2018; and W. L. Holland, et al., 2007; cited supra). Trends towards elevated hepatic C16:0 ceramide and muscle C18:0 ceramide levels were observed in HFD-fed mice (FIG. 21). Treating HFD-fed mice with sphingolipid-like compound 893 for 4 weeks restored normal triglyceride and ceramide levels in the liver and ceramide levels trended lower in muscle. Thus, consistent with genetic studies indicating that blocking mitochondrial fission can correct hepatic steatosis (L. Wang, et al., 2015; and D. Sebastien, et al., 2012; cited supra), sphingolipid-like compound 893 reversed the accumulation of toxic lipid species in HFD-fed mice.

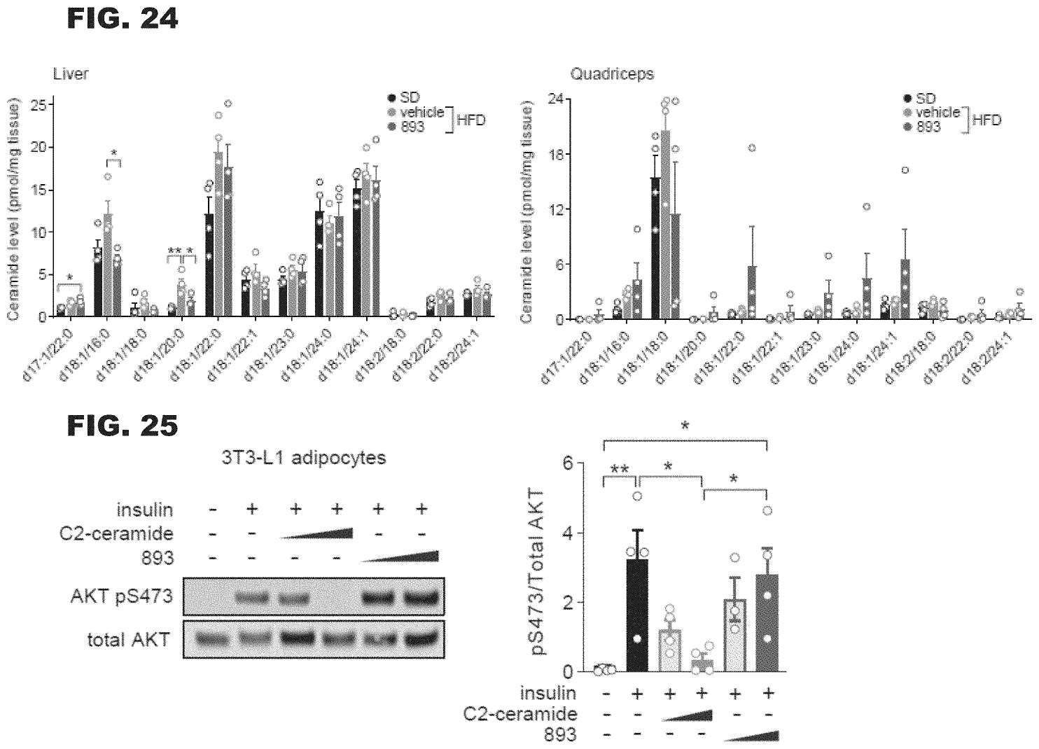

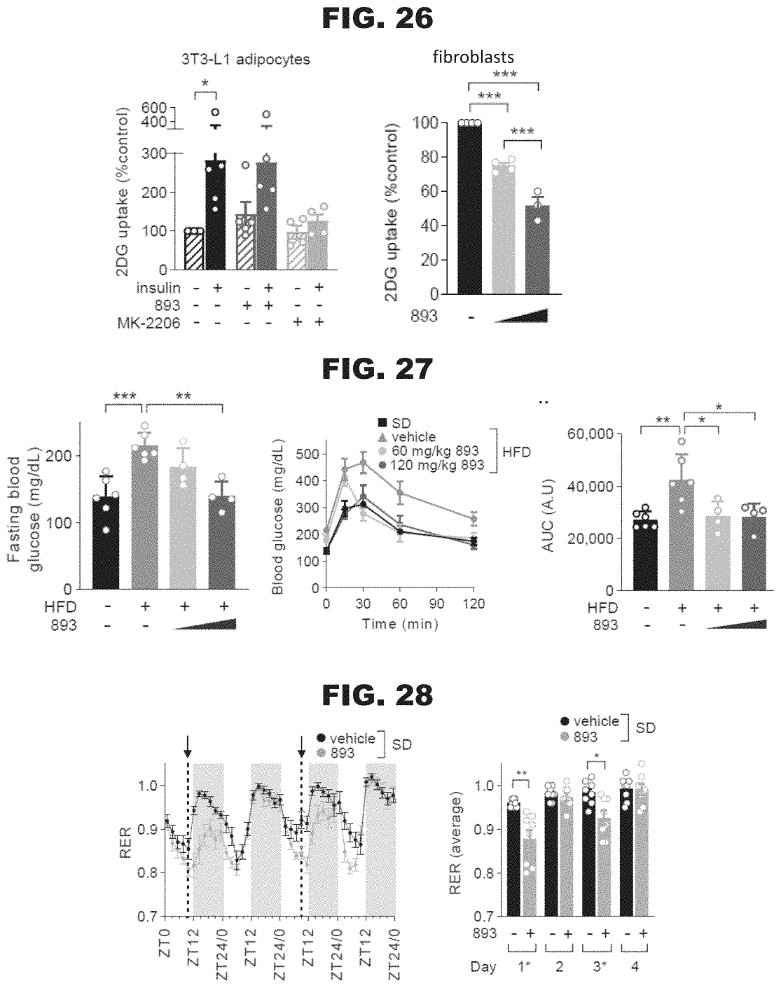

Insulin resistance is a hallmark of the metabolic syndrome. Ceramide disrupts insulin-dependent signaling by inducing mitochondrial fragmentation, but also by reducing AKT phosphorylation and thus GLUT4 translocation to the plasma membrane. Although sphingolipid-like compound 893 shares ceramide's ability to activate protein phosphatase 2A (PP2A), sphingolipid-like compound 893 does not reduce AKT activity (P. Kubiniok, et al., Mol. Cell Proteomics 18: 408-422, 2019, the disclosure of which is incorporated herein by reference; see also, S. M. Kim, 2016, cited supra). Indeed, ceramide, but not sphingolipid-like compound 893, interfered with insulin-stimulated AKT activation in 3T3-L1 adipocytes (FIG. 25). Consistent with this result, the AKT inhibitor MK-2206, but not sphingolipid-like compound 893, impeded insulin-stimulated glucose uptake in adipocytes (FIG. 26; constitutive glucose uptake in fibroblasts was reduced by sphingolipid-like compound 893 as expected given sphingolipid-like compound 893's ability to down-regulate GLUT1 (FIG. 26) (G. G. Guenther, et al., Oncogene 33: 1776-1787, 2014, the disclosure of which is incorporated herein by reference). Normalization of mitochondrial morphology without AKT inhibition suggested that treatment with sphingolipid-like compound 893 might restore insulin sensitivity in mice maintained on a HFD. Vehicle-treated mice fed the HFD for 12 weeks exhibited fasting hyperglycemia as expected (FIG. 27). However, treatment with 120 mg/kg sphingolipid-like compound 893 three days a week for 25 days normalized both fasting glucose and glucose disposal in HFD-fed mice as demonstrated by an oral glucose tolerance test (OGTT) (FIG. 27); the 60 mg/kg dose produced an intermediate effect. In sum, consistent with the restoration of normal mitochondrial morphology in the livers of HFD-fed mice, 12 doses of 120 mg/kg sphingolipid-like compound 893 over 4 weeks fully corrected the hepatic lipid accumulation, fasting hyperglycemia, and insulin resistance associated with continuous HFD feeding.

### Sphingolipid-Like Compound 893 Increases Body Fat Catabolism by Reducing Food Intake.

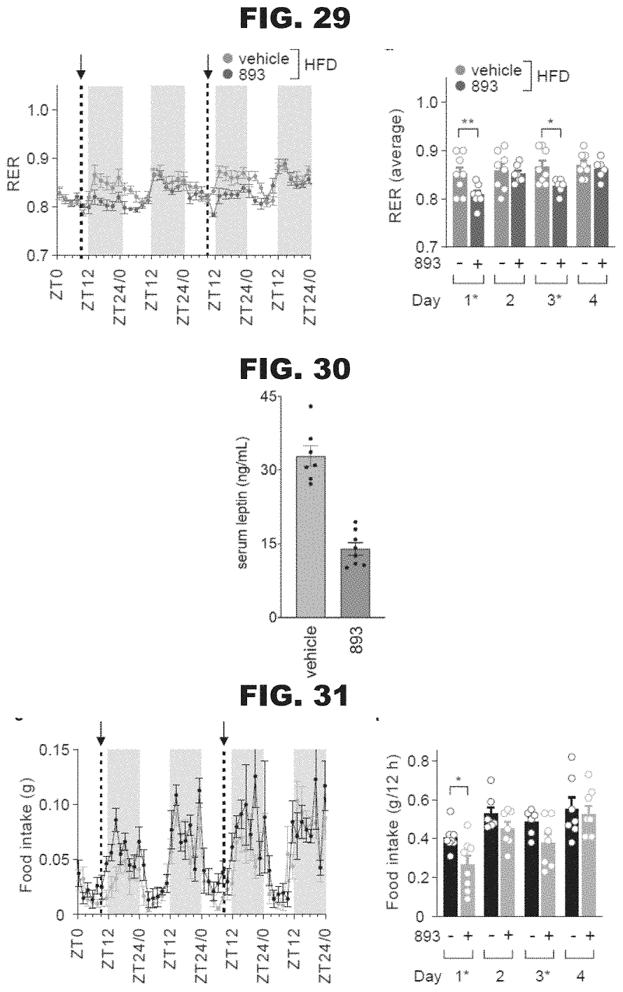

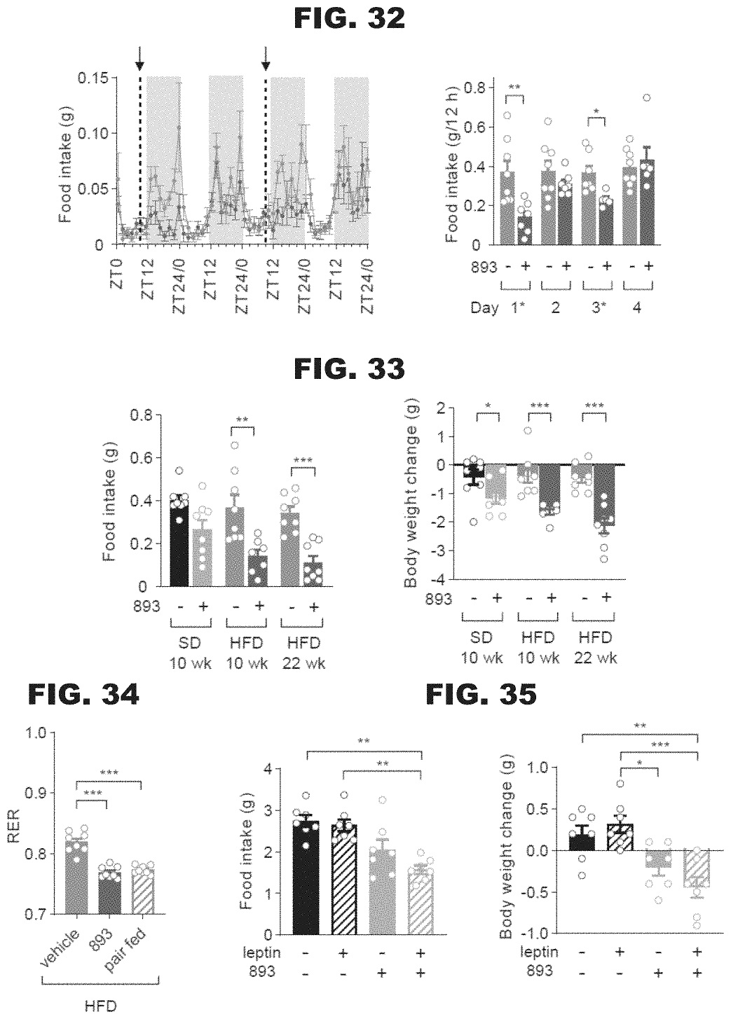

To examine the effects of sphingolipid-like compound 893 on whole body metabolism, indirect calorimetry was performed. Male C57BL/6J mice were maintained on the SD or HFD for 10-12 weeks, acclimated to metabolic cages that mimic the home environment, and then treated with vehicle or 120 mg/kg sphingolipid-like compound 893 by gavage at ZT8.5. The ratio between the amount of CO₂ produced and the O₂ consumed (respiratory exchange ratio (RER)) reflects relative whole-body fuel substrate utilization with a value close to 0.7 indicating that fat is primarily being utilized and a value of 1.0 indicating that carbohydrates are the main fuel source. As expected, RER values were lower during both light and dark cycles and diurnal fluctuations in substrate utilization were blunted in HFD-fed mice relative to SD controls (FIGS. 29 & 30). Treatment with sphingolipid-like compound 893 reduced the RER in both HFD- and SD-fed mice. Consistent with the pharmacokinetic properties of sphingolipid-like compound 893 (see FIG. 12), RER returned to control values 24 h after sphingolipid-like compound 893 treatment (FIGS. 28 & 29). Animals were similarly responsive to a second treatment with sphingolipid-like compound 893 given 48 h after the first. In contrast with the clear reduction in RER, energy expenditure calculated using the Weir formula was not significantly affected by sphingolipid-like compound 893. A trend towards reduced activity as measured by XY beam breaks may relate to decreased food seeking behavior given the equivalent use of running wheels by vehicle- and sphingolipid-like compound 893-treated mice. In summary, indirect calorimetry revealed that sphingolipid-like compound 893 increases the utilization of fat without significantly affecting activity or energy expenditure.

Further assessment was performed to determine the level of leptin circulating in HFD-fed mice treated with sphingolipid-like compound 893. Mice were fed a HFD for 4 weeks and randomly distributed into two groups with the same mean weight. Mice were gavaged with water (n=7) or 120 mg/kg 893 (n=8) at ZT8.5. Blood was collected from the saphenous vein between ZT15-18 in pairs of treated vs. untreated animals and leptin levels were measured in serum using ELISA (CrystalChem, cat #90030). Sphingolipid-like compound 893-treated animals had lowered levels of circulating leptin (FIG. 30).

If sphingolipid-like compound 893-treated animals are losing weight but not expending more energy, fewer calories must be available to metabolically active tissues. Reversing mitochondrial fragmentation in the hypothalamus (see FIG. 14) and reducing circulating leptin levels (see FIG. 30) should suppress appetite. In fact, continuous monitoring in metabolic caging revealed that sphingolipid-like compound 893 reduced food intake (FIGS. 31 & 32). This trend was apparent in both mice fed the SD and in mice maintained on the HFD for 10-12 weeks although the effect was more pronounced in the HFD group. When these studies were repeated in mice maintained on the HFD for 22 weeks, the suppression of food intake and body weight loss appeared to increase (FIG. 33). To determine whether the reduction in food consumption was sufficient to account for the suppression of RER by sphingolipid-like compound 893, a paired feeding study was performed. Providing untreated, HFD-fed mice with only the reduced amount of food eaten by sphingolipid-like compound 893-treated mice between ZT12 and ZT24 resulted in an equivalent reduction in RER (FIG. 34). Consistent with this result, normalizing food intake by gavaging chow-fed mice at ZT12 with a liquid diet containing the number of calories consumed from ZT12-ZT15 by lean, vehicle control mice eliminated the effect of sphingolipid-like compound 893 on the RER. When access to solid food was restored at ZT17, a trend towards reduced RER was again observed. When administered in the morning at ZT2 rather than in the afternoon at ZT8.5, sphingolipid-like compound 893 still decreased both food intake and RER although statistical significance was not achieved during the light period, most likely because sphingolipid-like compound 893 levels peaked when mice were inactive and food intake was low. Thus, the reduced RER in sphingolipid-like compound 893-treated mice likely stems from reduced carbohydrate availability and increased utilization of fat stores rather than from primary changes in how dietary components are metabolized.

Leptin is secreted by adipocytes in proportion to their triglyceride content, signaling to the CNS when peripheral energy stores are full and food consumption should decrease. In HFD-fed mice, chronic increases in adiposity lead to elevations in circulating leptin with no accompanying decrease in food intake, a state that has been termed leptin-resistant. Reducing leptin levels in the blood restores leptin signaling in the hypothalamus (S. Zhao, et al., 2019, cited supra). Thus, the sphingolipid-like compound 893 should function as a leptin-sensitizing agent. To test this model, 18 week old lean, male C57BL/6J mice were treated intraperitoneally with a suboptimal dose of recombinant leptin at ZT8.5 and food intake and body weight measured over the next 18 h. As expected, peripheral administration of 2 mg/kg leptin was not sufficient to decrease food intake or body weight in these mice (FIG. 35). Consistent with metabolic cage studies, sphingolipid-like compound 893-treatment produced a trend towards reduced food intake and body weight in chow-fed mice maintained in standard caging (FIG. 35). Intriguingly, combining the ineffective dose of leptin with sphingolipid-like compound 893 was sufficient to produce a statistically significant decrease in food intake and body weight. In summary, the reduction in food intake in sphingolipid-like compound 893-treated mice is consistent with the reversal of mitochondrial fragmentation in the hypothalamus and likely due in part to the re-sensitization of anorexigenic POMG neurons to leptin when plasma leptin levels are decreased.

### Mitochondrial Fragmentation Drives Metabolic Dysfunction in HFD-Fed but not Leptin-Deficient Mice.

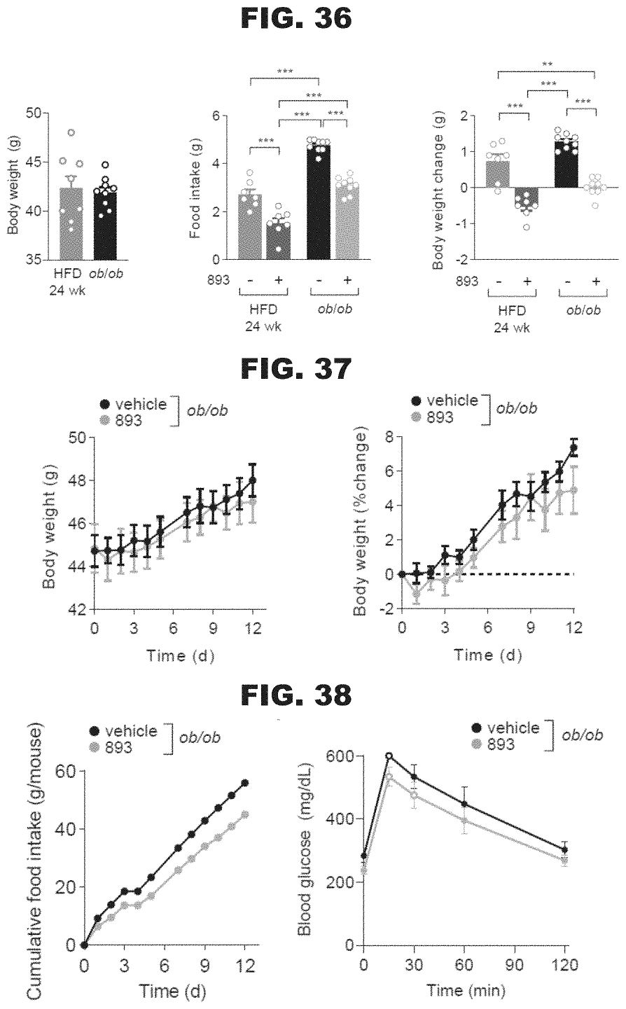

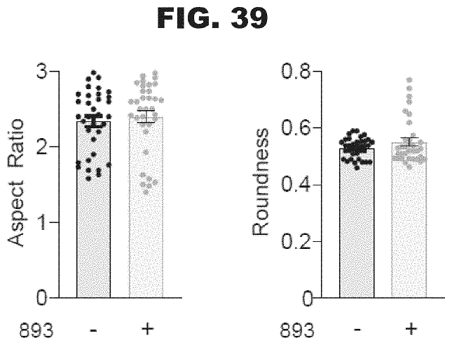

To more rigorously assess the role of leptin in the anti-obesogenic effects of sphingolipid-like compound 893, the response of leptin-deficient ob/ob mice to sphingolipid-like compound 893 was measured. As expected, ob/ob mice were hyperphagic and became obese on the standard chow diet provided by University Lab Animal Resources (16% kcal from fat). Ob/ob animals were used in experiments once they attained an equivalent body weight to C57BL/6J mice fed the HFD for 24 weeks (FIG. 36). Even in the absence of leptin, sphingolipid-like compound 893 decreased food intake (FIG. 36). However, sphingolipid-like compound 893-treated ob/ob mice still consumed more food than treated wild type, HFD-fed controls suggesting a role for leptin in the anorexigenic actions of sphingolipid-like compound 893 (FIG. 36). Strikingly, repeated dosing with sphingolipid-like compound 893 failed to produce weight loss in ob/ob mice as it did in wild type mice. While six doses of 120 mg/kg sphingolipid-like compound 893 over 2 weeks reduced the body weight of HFD-fed wild type mice by 10% (FIG. 16), sphingolipid-like compound 893-treated ob/ob mice exhibited a 5% weight gain over the same interval (FIG. 37). Moreover, repeated dosing with sphingolipid-like compound 893 produced only a modest decrease in cumulative food intake in ob/ob mice (FIG. 38). sphingolipid-like compound 893 also failed to correct fasting hyperglycemia or restore glucose tolerance in mice lacking leptin (FIG. 38) as it did in HFD-fed wild type mice (see FIG. 27). In summary, sphingolipid-like compound 893 slightly reduced food intake but failed to produce weight loss or correct obesity-associated metabolic defects in leptin-deficient ob/ob mice as it did in HFD-fed, wild type animals.

These studies demonstrate that the synthetic sphingolipid-like compound 893 prevents mitochondrial fragmentation in response to ceramide and other signals more effectively, potently, and/or rapidly than other agents reported to modulate mitochondrial morphology. In keeping with its robust in vitro effects and favorable pharmacologic properties, sphingolipid-like compound 893 acutely restored normal mitochondrial morphology in HFD-fed mice, increasing aspect ratio and reducing roundness in both liver and brain mitochondria after a single dose. These effects on mitochondrial shape are sufficient to explain the constellation of beneficial outcomes observed in mice consuming a HFD and treated with sphingolipid-like compound 893: reduced plasma leptin, reduced food intake, improved glucose tolerance, and the resolution of hepatic steatosis. Triggering mitochondrial fragmentation in adipocytes by deleting Mfn2 is sufficient to elevate leptin levels, increase food intake, and induce obesity. Thus, 893 likely corrects hyperleptinemia in obese mice by increasing mitochondrial tubularity in white adipocytes. Limiting mitochondrial fission in the liver by deleting DRP1 or expressing a dominant-negative DRP1 mutant increases insulin sensitivity, reduces weight gain, and corrects hepatic steatosis in mice on a HFD. Conversely, promoting mitochondrial fission in the liver by reducing the expression of the mitochondrial fusion factor MFN2 leads to insulin resistance. Blocking mitochondrial fission in anorexigenic POMC neurons by deleting DRP1 or over-expressing MFN2 sensitizes to leptin and reduces food intake. Conversely, promoting fission in POMC neurons by knocking out MFN2 produces leptin resistance and hyperphagia. The finding that sphingolipid-like compound 893 reduced food intake in SD-fed mice is also consistent with published studies showing that deleting DRP1 from POMC neurons limits food intake in chow-fed mice where mitochondrial dynamics are not basally perturbed. Thus, the ability of 893 to oppose mitochondrial fission in adipocytes, liver, brain and likely other metabolic tissues is sufficient to account for its beneficial effects in HFD-fed mice.

The failure of sphingolipid-like compound 893 to produce weight loss or improve glucose handling in ob/ob mice provides additional evidence in favor of a mitochondrial mechanism of action; these leptin-deficient mice have hypertubulated mitochondria despite elevated C16:0 ceramide levels. This finding might be explained by the starvation-like state created by leptin deficiency and the ability of starvation to promote mitochondrial fusion. The mild decrease in food intake in sphingolipid-like compound 893-treated ob/ob animals is necessarily leptin-independent, but still consistent with the proposed mitochondrial mechanism of action. Mitochondrial tubulation in adipocytes would also be expected to increase adiponectin secretion, an anoerexigenic hormone. In addition, mitochondrial fragmentation reduces insulin sensitivity in multiple tissues. Like leptin, glucose and insulin trigger αMSH secretion from POMC neurons; inhibiting mitochondrial fission likely sensitizes anorexigenic POMC neurons to insulin and/or glucose as well as leptin. Although studies in obese and diabetic patients often measure mitochondrial function without interrogating mitochondrial morphology, increased mitochondrial fission has been linked to increased adiposity and metabolic dysfunction in humans as well as mice. Patients homozygous for a missense mutation in MFN2 have fragmented, spherical adipocyte mitochondria and a dramatic upper body adipose tissue over-growth syndrome. Large, round mitochondria have also been observed in pancreatic β cells of patients with type 2 diabetes. In summary, the ability of sphingolipid-like compound 893 to reverse mitochondrial fragmentation is sufficient to explain its beneficial effects on the body weight and metabolism of HFD-fed mice.

Therapeutics that modulate mitochondrial function are highly sought after. While agents that alter mitochondrial morphology have been previously reported, sphingolipid-like compound 893 is more effective, more potent, and/or works more rapidly in vitro, and sphingolipid-like compound 893 completely corrects obesity and metabolic dysfunction in HFD-fed mice. As reported by others and consistent with our findings, mdivi-1 does not directly inactivate mammalian DRP1. While combining mdivi-1 with the putative fusion promoter M1 was reported to produce more tubulated mitochondrial networks in T cells, neither compound alone or in combination prevented ceramide- or KRAS-induced mitochondrial fragmentation. Consistent with this lack of effect on mitochondrial dynamics, mdivi-1's benefits in obesity models are limited in scope, and a reduction in food intake has not been reported. Like mdivi-1, the diabetes treatment metformin is a mitochondrial complex 1 inhibitor, and the benefits of mdivi-1 may be related to this activity rather than changes in mitochondrial dynamics. The peptide P110 that mimics a putative protein-protein interaction domain in DRP1 did prevent ceramide-induced mitochondrial fragmentation, but only after a prolonged incubation. Although P110 has been tested in mouse models of neurodegenerative disease, no published reports document activity in obesity models. Even if P110's pharmacokinetic properties prove adequate to protect from a HFD, an orally-bioavailable small molecule like sphingolipid-like compound 893 would likely have superior value as an obesity therapeutic given that a peptide drug must be administered parenterally and chronic treatment would like be required. The orally-active, FDA-approved anti-inflammatory leflunomide that up-regulates MFN2 also blocked ceramide-induced mitochondrial fragmentation in vitro. However, leflunomide was 10-fold less potent than sphingolipid-like compound 893, required a prolonged pre-incubation, and its effects were more context dependent (e.g. FIG. 9). Moreover, as for mdivi-1, leflunomide's modest therapeutic value in obese mice is likely to be independent of effects on mitochondrial dynamics. Leflunomide's effects on glucose metabolism were more significant in ob/ob than in HFD-fed mice, while the results described herein clearly demonstrate that sphingolipid-like compound 893 is more effective in HFD-fed animals. Leflunomide up-regulates MFN2 by inhibiting dihydroorotate dehydrogenase, and its effects on mitochondrial morphology can be reversed by uridine supplementation which allows pyrimidine synthesis via the salvage pathway. In obese mice, supplementation with uridine did not undermine leflunomide's effects on glucose metabolism suggesting an alternative mechanism of action. Finally, leflunomide failed to reduce food intake or body weight in obese mice as would be predicted for an agent that reverses mitochondrial fragmentation. Notably, mitochondrial morphology was not assessed in obese mice treated with leflunomide. Even though leflunomide is FDA-approved, it can have serious toxicities. Following fatal liver failure in 14 rheumatoid arthritis patients taking the drug, the FDA issued a Boxed Warning for leflunomide indicating that it should not be taken by patients with pre-existing liver disease. Chronic treatment with the high dose of sphingolipid-like compound 893 was not hepatotoxic in otherwise healthy tumor-bearing mice, although sphingolipid-like compound 893's toxicity in the context of liver disease will need to be evaluated. In summary, this report defines sphingolipid-like compound 893 as the most robust inhibitor of mitochondrial fission identified to date and provides the first demonstration that pharmacologic reversal of mitochondrial fragmentation is highly effective, resolves hyperleptinemia, and is well tolerated in mice with HFD-induced obesity.

### Materials and Methods

### General Animal Procedures

All animal experiments were performed in accordance with the Institutional Animal Care and Use Committee of University of California, Irvine. Male mice were used in all experiments. C57BL/6J mice (stock no 000664) and ob/ob mice (stock no 000632) were purchased from the Jackson Laboratory and were acclimated for 7 days prior to beginning experiments. Mice were housed under a 12:12 h light-dark cycle at 20-22° C. in groups of 4-5. Cages contained ⅛″ corncob bedding (7092A, Envigo, Huntingdon, UK) enriched with ˜6 g of cotton fiber nestlets (Ancare Corp., Bellmore, N.Y.). Access to food and water was ad libitum unless otherwise specified. For HFD studies, 8 week old C57BL/6J males were randomly assigned to either a 45% kcal from fat diet (HFD; D12451, Research Diets Inc., New Brunswick, N.J.) or 10% kcal from fat diet designed to match D12451 for other components (SD; D12450B, Research Diets Inc). Mice were maintained on these diets for up to 22 weeks as indicated. Ob/ob mice were fed the vivarium stock diet which contained 16% kcal from fat (2020×, Envigo). Polypropylene feeding tubes (20 g×38 mm; Instech Laboratories Inc., Plymouth, Pa.) were utilized for gavage and dipped into a 1 g/ml sucrose solution immediately prior to treatment to induce salivation.

### Weight Loss Intervention Study

HFD-fed mice were randomly assigned to experimental groups receiving either vehicle (water) or sphingolipid-like compound 893 at 60 mg/kg or 120 mg/kg by oral gavage on Mondays, Wednesdays, and Fridays. Mice were maintained on the HFD throughout the study. The SD group was treated with vehicle on the same schedule. Group size was initially n=10; one animal from the 60 mg/kg group that was euthanized due to gavage error was excluded from the analysis making this group n=9. All treatments and measurements were performed between ZT8 and ZT10 unless otherwise noted. Body weight and food consumption were monitored Monday-Friday. Body composition was determined weekly in live animals using an EchoMRI™ Body Composition Analyzer (EchoMRI™ Corp., Singapore). Mice were euthanized and tissues collected 4 h after treatment. Where indicated, mice were fasted for 6 h.

### Voluntary Cage Running

To monitor voluntary exercise, sixteen mice were singly housed in home cages equipped with running wheels; initially n=8. A magnet was affixed to each 240 mm wheel and a bicycle odometer (Sigma BC509, Sigma Sports, Chicago) used to count the number of wheel revolutions and time spent on running on the wheels. Distance run was calculated by the equation (#revolutions×running wheel circumference=distance). The wheels were cleaned and randomly re-assigned weekly to each cage to control for differences in wheel performance. Two animals in the sphingolipid-like compound 893-treated group were euthanized due to gavage errors and were excluded from the analysis resulting in n=6.

### Blood Glucose Measurements and Oral Glucose Tolerance Tests (OGTT)

Mice were fasted for 6 h prior to blood glucose testing at ZT10. When sphingolipid-like compound 893 treatment was combined with an OGTT, mice were treated at ZT6. Once baseline fasting blood glucose was determined using a handheld blood glucose meter (Prodigy Diabetes Care, Charlotte, N.C.) and a drop of blood collected from a tail vein nick, mice were gavaged with an oral glucose solution (20% w/v in water, 2 g/kg bodyweight) and blood glucose measured in a drop of tail vein blood at 0, 15, 30, 60 and 120 min. The area under the curve was determined using Graphpad Prism software.

### Indirect Calorimetry

Metabolic parameters were measured using the Phenomaster system (TSE Systems Inc., Chesterfield, Mo.). The climate chamber was set to 21° C. and 50% humidity with a 12:12 h light-dark cycle. Mice were singly housed inside the chamber and acclimated for 48 h prior to data collection. VO₂, VCO₂, and food intake was measured every 27 min. Respiratory exchange ratio (RER) was calculated using the formula RER=VCO₂/VO₂. Energy expenditure was calculated using the equation EE=1.44(3.941×VO₂+1.106×VCO₂). In the indirect calorimetry studies in FIGS. 12 to 14, C57BL/6J mice were maintained on the HFD or SD for 10-12 weeks prior to evaluation and were naïve to therapy. Mice were serially evaluated in cohorts of 4 vehicle- and 4 sphingolipid-like compound 893-treated mice using 8 metabolic cages. Treatment was by gavage at ZT8.5, sphingolipid-like compound 893 was administered at 120 mg/kg in water. To evaluate the effect of morning treatment, vehicle or 120 mg/kg sphingolipid-like compound 893 was administered at ZT2 to 18 week old C57BL/6J males on a standard diet. Liquid diet experiments were also performed on 18 week old C57BL/6J males; mice were treated with sphingolipid-like compound 893 at ZT8.5 and food access was restricted at ZT11. Liquid feed (AIN-76, BioServ, Flemington, N.J.) was prepared at 1000 kcal/L in milli-Q water and at ZT12, mice were gavaged with 400 μL (0.4 kcal) of diet, corresponding to approximately 3 h of ad libitum consumption of standard chow. For the pair feeding study, RER was monitored over 12 h in mice maintained on the HFD for 22 weeks (31 weeks of age); pair-fed mice were used after a 48 h wash-out period and provided with the average amount of food they ate over 24 h after sphingolipid-like compound 893 treatment (92.5 mg or 0.4 kcal).

Data was excluded from these analyses as follows. During measurements of one SD cohort (4 vehicle- and 4 sphingolipid-like compound 893-treated mice), a leak in the reference CO₂ system was detected, the O₂ and CO₂ data was censored until the leak was corrected (70-74 h). Feeding and activity measurements were not compromised and were still analyzed. Occasionally, uneaten food was found on the floor of the cage precluding use of the hopper sensor to accurately monitor food intake. In these instances, food intake data was censored for the prior 24 h period (days 2 and 3 for mouse 8 (SD+sphingolipid-like compound 893) and mouse 8 (SD+vehicle), and day 3 for mouse 8 (HFD+sphingolipid-like compound 893) and mouse 1 (HFD+vehicle)). A sensor malfunction due to the mouse dislodging the hopper also resulted in the exclusion of food intake data (mouse 5 (SD+vehicle) days 2-4). In rare cases, inadvertent pharyngeal administration of gavage material occurred. These mice were not euthanized, but food intake, calorimetry, and activity data from these animals was excluded from the analysis for 1 week after this event (mouse 2 (HFD+sphingolipid-like compound 893) after the second treatment on day 3 and mouse 5 (HFD+sphingolipid-like compound 893) after the first dose on day 1).

### Home-Cage Feeding Studies

Mice were singly housed and allowed to acclimate for 72 h before food intake was monitored. Food consumption was determined by monitoring the weight of food in the hopper. Initial food and body weight measurements were taken at ZT9 and final measurements were taken 16 h later to capture the active period where most consumption occurred. Home cage feeding studies were performed with C57BL/6J mice maintained on a HFD for 24 weeks (33 weeks of age), or ob/ob mice at 8 weeks of age. Mice received vehicle or 120 mg/kg sphingolipid-like compound 893 by gavage at ZT8.5. For experiments involving leptin, 18 week old, SD fed (16% kcal from fat, 2020×, Envigo) C57BL/6J mice received vehicle or 120 mg/kg sphingolipid-like compound 893 by gavage at ZT8.5. At ZT11.5, vehicle (20 mM Tris-Cl, pH 8.0) or 2 mg/kg recombinant mouse leptin (498-OB, R&D Systems, Minneapolis, Minn.) was delivered by intraperitoneal injection. The same 8 mice were used for all treatments following a 48 h washout period, and treatments were administered in the following order: vehicle, sphingolipid-like compound 893, leptin, and leptin+sphingolipid-like compound 893. All data collected from one mouse was excluded due to inadvertent pharyngeal administration during gavage reducing the n from 8 to 7 (FIG. 5a-d). One ob/ob mouse that failed to gain weight on the chow diet (bodyweight >20% less than littermates) was excluded from all analyses. One ob/ob mouse died during Echo MRI for unknown reasons after 6 d of treatment with sphingolipid-like compound 893; data from this mouse was analyzed prior to death.

### Lipidomic Profiling

Lipids were extracted from liver and quadriceps tissue using a modified MTBE method (Matyash et al, 2008; Abbott et al, 2013). Briefly, 10 mg/ml of tissue was homogenized in ice-cold 150 mM ammonium acetate using a bead homogenizer (1.4 mm ceramic) kept below 4° C. using liquid nitrogen vapor (Precellys 24 homogenizer with Cryolys cooling unit, Bertin Technologies, Montigny-le-Bretonneux, France). From this, 20 μl of homogenized tissues were added to glass vials containing MTBE and methanol (3:1 v/v, with 0.01% BHT), alongside 10 μl of an internal standard solution containing 10 μM each: phosphatidylcholine (PC) 17:0/17:0, phosphatidylethanolamine (PE) 17:0/17:0, phosphatidylserine (PS) 17:0/17:0, phosphatidylglycerol (PG) 17:0/17:0, lysophosphatidylcholine (LPC) 17:0, lysophosphatidylethanolamine (LPE) 14:0, ceramide (Cer) d18:1/17:0, dihydrosphingomyelin d18:0/12:0, diacylglycerol (DAG) 17:0/17:0, D5-triacylglycerol (TAG) 48:0, and cholesteryl ester (CE) 22:1. Samples were allowed to rotate at 4° C. overnight prior to the addition of 1 volume of ice-cold 150 mM ammonium acetate. Samples were vortexed thoroughly prior to centrifugation (2000×g, 5 min) to enable phase separation. The upper organic phase was removed to a new vial and dried under a stream of nitrogen with gentle heating (37° C.). The dried lipids were reconstituted in chloroform:methanol:water (60:30:4.5 v/v/v) and kept at −20° C. until analysis.

Extracted lipids were analyzed by liquid chromatography-mass spectrometry (LC-MS) using a Dionex Ultimate 3000 LC pump and Q Exactive Plus mass spectrometer equipped with a heated electrospray ionization (HESI) source (Thermo Fisher Scientific). Lipids were separated on a Water ACQUITY C18 reverse phase column (2.1×100 mm, 1.7 μm pore size, Waters Corp., Milford, Mass.) using a binary gradient, where mobile phase A consisted of acetonitrile:water (6:4 v/v) and B of isopropanol:acetonitrile (9:1 v/v). Both mobile phases A and B contained 10 mM ammonium formate and 0.1% formic acid, the flow rate was 0.26 ml/min, and the column oven was heated to 60° C. Source conditions were as follows: a spray voltage of 4.0 and 3.5 kV in positive and negative ion modes respectively, capillary temperature of 290° C., S lens RF of 50, and auxiliary gas heater temperature of 250° C. Nitrogen was used as both source and collision gas, with sheath and auxiliary gas flow rate set at 20 and 5 (arbitrary units) respectively. Data were acquired in full scan/data-dependent MS2 mode (full scan resolution 70,000 FWHM, max ion injection time 50 ms, scan range m/z 200-1500), with the 10 most abundant ions being subjected to collision-induced dissociation using an isolation window of 1.5 Da and a normalized stepped collision energy of 15/27 eV, with product ions detected at a resolution of 17,500. An exclusion list for background ions was developed using extraction blanks, and mass calibration was performed in both positive and negative ionization modes prior to analysis to ensure mass accuracy of 5 ppm in full scan mode.

Lipids were analyzed using MS-DIAL (Tsugawa et al, 2015). Lipids were detected in both positive and negative ionization modes using a minimum peak height of 1×10⁴ cps, a MS1 tolerance of 5 ppm and MS2 tolerance of 10 ppm, and a minimum identification score of 50%. Identified peaks were aligned with a retention time tolerance of 0.5 min. Exported aligned data were background subtracted and quantified from internal standards using the statistical package R. One-way ANOVA with Tukey post-hoc analysis was used to identify differences between groups with statistical significance set at an adjusted P<0.05.

### Targeted Metabolite Quantification

Plasma pharmacokinetic analysis of sphingolipid-like compound 893 was performed by Pharmaron Corporation (Beijing, China). C16:0 ceramide levels were quantified in cells using the method described in (T. Kasumov, et al., Anal. Biochem. 401: 154-161, 2010, the disclosure of which is incorporated herein by reference) with minor modifications. Cultured cells were washed twice in PBS and scraped into 250 μL of HPLC grade water and flash frozen until time of analysis. On the day of analysis, samples were thawed, and an aliquot used for protein quantification. For C16:0 ceramide levels in mouse liver, 25 mg of tissue was homogenized in 1 ml of ice-cold PBS using a mechanical probe homogenizer (VWR, Radnor, Pa.), protein levels quantified, and 50 μl of the homogenate diluted with 150 μl HPLC-grade water for C16:0 ceramide analysis. Fifty ng of C17:0 ceramide prepared in ethanol (#22532, Cayman Chemical, Ann Arbor, Mich.) was added into 200 μL of the thawed cell suspension or liver homogenate as an internal standard to control for varying extraction efficiency; 750 μL of an ice-cold 1:2 chloroform/methanol mixture was then added. Samples were sonicated for 30 min and phase separation induced by the addition of 250 μL each of chloroform and HPLC-grade water. Samples were centrifuged at 4° C. for 10 min and the lower lipid phase transferred to a clean tube. The remaining protein and aqueous layers were re-extracted with an additional 500 μL of chloroform. Lipid phases were combined and then dried under vacuum. Dried extract was re-constituted in 100% acetonitrile immediately before analysis. Samples were analyzed by ultra-performance liquid chromatography tandem mass spectrometry (UPLC-MS/MS) using a Waters Micromass Quattro Premier XE equipped with a Waters ACQUITY BEH C4 column (Waters Corp.). Samples were resolved starting at 60% mobile phase A (10 mM ammonium acetate and 0.05% formic acid in water) to 98% mobile phase B (60:40 acetonitrile:isopropanol) over 3 min with a linear gradient, held at 98% B for 1 min, then the column was equilibrated with 60% A for 1 min. The mass spectrometer was operated in positive ion mode with the following parameters: cone voltage 20 V, source temperature 125° C., desolvation temperature 400° C. Ion transition channels for MS/MS were 538→264 for C16:0 ceramide and 552→264 for C17:0 ceramide, both with a dwell time of 285 ms. Standard curves prepared from C16:0 ceramide (#860516, Avanti Polar Lipids, Alabaster, Ala.) dissolved in ethanol were used for quantitation and were linear from 4.1 nM-1,000 nM, with an R² of 0.98 or greater.

### Cell Culture

3T3-L1 cells were maintained in DMEM with 10% FBS and 1% penicillin-streptomycin until induced to differentiate. 3T3-L1 pre-adipocytes were differentiated as described in (M. P. Valley, et al., Anal. Biochem. 505: 43-50, 2016, the disclosure of which is incorporated herein by reference) with slight modifications. Briefly, pre-adipocytes were grown to confluence. After 2 d, cells were induced with maintenance media containing 500 μM IBMX (15879, Sigma-Aldrich, St. Louis, Mo.), 1 μM dexamethasone (D4902, Sigma-Aldrich), 5 μg/mL bovine insulin (10516, Sigma-Aldrich), and 5 μM troglitazone (50-115-0786, ApexBio). Media was changed every 2-3 d for 7 d. On day 7 post-induction, media was changed to maintenance media+5 μg/mL bovine insulin. On day 9 post-induction, media was changed to maintenance media. Mature adipocytes were used 12-14 d post-induction for all experiments. LSL-KrasG12D mouse embryonic fibroblasts (MEFs) with and without Cre-mediated deletion of the STOP cassette were obtained from David Tuveson (Cold Spring Harbor Laboratory, Cold Spring Harbor N.Y., USA) in 2000. p53^{flox/flox} MEFs were derived in house (2015) from C57BL/6 mice using standard techniques and immortalized by transient expression of Cre recombinase and deletion of p53. MEFs were cultured and maintained in DMEM with 10% FBS and 1% penicillin-streptomycin. A549 cells were cultured in DMEM with 10% FBS, 1% penicillin-streptomycin and 1% sodium pyruvate. Stock solution of palmitic acid (ACROS Organics, cat # AC129702500) was prepared at 100 mM in ethanol. Palmitate (250 μM) was conjugated to 1% (w/v) fatty-acid free bovine serum albumin (Sigma, A8806) in DMEM at 37° C. for 20 min. For all immunofluorescence assays, 8,000 MEFs were seeded into 8-chamber slides (Cellvis, cat # C8-1.5H-N) 12-16 h before treatment. Cells were pre-treated with sphingolipid-like compound 893 (5 μM in water), myriocin (10 μM in methanol), fumonisin-B1 (30 μM in DMSO), or celastrol (500 nM in DMSO) for 3 h followed by a 3 h treatment with BSA-conjugated palmitate mixture or BSA alone. Where cells were treated with C2-ceramide (50 μM in DMSO) or C16:0 ceramide (100 μM in ethanol) for 3 h, cells were pre-treated with sphingolipid-like compound 893 for 1-3 h as indicated, mdivi-1 (50 μM in DMSO) for 1 h or 24 h, M1 (5 μM in DMSO) for 24 h, mdivi-1 and M1 together for 24 h, leflunomide (50 μM in methanol) for 1 h or 24 h, or with P110 (1 μM in water) for 1 h or 12 h. LSL or KRAS^{G12D} MEFs were treated with sphingolipid-like compound 893 (5 μM) for 6 h prior to fixation.

### Western Blot Analysis

Mature adipocytes were serum starved for 16 h then treated with vehicle, ceramide (50 or 100 μM), or sphingolipid-like compound 893 (5 or 10 μM) in serum-free media supplemented with 0.2% fatty-acid free BSA for 3 h after which 100 nM insulin was added for 15 min. To determine total DRP1 protein levels, 100,000 MEFs were seeded into a 6-well plate for 16 h, pre-treated for 3 h with vehicle or sphingolipid-like compound 893 (5 μM) followed by a 3 h incubation in 1% BSA+ethanol or BSA-palmitate (250 μM). Cells were washed once with cold PBS, then lysed in cold RIPA buffer (140 mM NaCl, 10 mM Tris pH 8.0, 1% Triton X-100, 0.1% SDS, 1% sodium deoxycholate) with cOmplete™ protease inhibitor (Cat no. 11697498001, Millipore Sigma, St. Louis, Mo.) and phosSTOP™ phosphatase inhibitor (Cat no. 4906837001, Millipore Sigma). Samples were incubated on ice for 10 min and insoluble material removed by centrifugation (9000×g for 10 min at 4° C.). Protein content was quantified in the supernatant using the Pierce™ BCA Protein Assay Kit (Thermo-Fisher Scientific, Waltham, Mass.). Equal amounts of protein were prepared in NuPAGE® LDS Sample Buffer (NP0007, Invitrogen) containing 50 mM DTT, and heated at 70° C. for 10 min. Proteins were resolved on a NuPAGE® 4-12% Bis-Tris protein gel (NP0336, Invitrogen, Carlsbad, Calif.) and subsequently transferred to a nitrocellulose membrane. Membranes were blocked in 5% BSA in TBST for 1 h, then probed with primary antibodies overnight at 4° C. Antibodies used were rabbit-anti-AKT pS473 at 1:1,000 (#4058, Cell Signaling Technology, Danvers, Mass.), rabbit-anti-AKT at 1:1,000 (#4685, Cell Signaling Technology), rabbit-anti-DRP1 at 1:1,000 (#8570, Cell Signaling Technology), and mouse anti-tubulin at 1:10,000 (T8328, Millipore Sigma, St. Louis, Mo.). Blots were then washed 3× in TBST and incubated in 800CW-conjugated goat anti-rabbit (#926-32211, Li-COR, Lincoln, NB) and 680LT-conjugated goat anti-mouse (#925-68020, Li-COR) secondary antibodies at 1:10,000 in 5% BSA in TBST for 1 h. Blots were washed then imaged using a Li-COR Odyssey CLx instrument. Band intensity was quantified using Image Studio Lite V5.2 software (Li-COR).

### Glucose Uptake Assays

Glucose uptake assays were performed using the Glucose-Glo™ uptake Kit according to manufacturer's instructions (cat # J1342, Promega, Madison, Wis.). For basal glucose uptake in MEFs, cells were plated the night before in 96-well black, clear-bottom plates. Cells were treated for 3 h, washed once in PBS, then pulsed with 1 mM 2-DG in glucose-free media containing their respective drug treatments. After 10 min, the reaction was quenched and developed according to manufacturer's protocol. To assay insulin-stimulated glucose uptake, mature adipocytes in 96-well black clear-bottom plates were serum starved for 16 h. Cells were treated in serum-free media supplemented with 0.2% fatty-acid free BSA for 3 h. Cells were washed once in PBS and incubated in glucose-free media with their respective drug treatments, with or without 100 nM bovine insulin, for 15 min. A concentrated 2-DG stock was added directly to wells for 10 min (1 mM final concentration), then the reaction stopped and developed according to manufacturer's protocol.

### Microscopy

MEFs were washed twice with PBS and fixed with 4% paraformaldehyde for 10 min at RT. Cells were permeabilized with 0.3% Triton X-100 in blocking buffer containing 10% fetal bovine serum for 20 min at 37° C. followed by overnight incubation with mouse anti-citrate synthase (sc-390693, Santa Cruz Biotechnology; dilution, 1:200) or rabbit-anti-DRP1 at 1:100 (#8570, Cell Signaling Technology) at 4° C. Cells were then washed twice with PBS and incubated with AlexaFluor 488 goat anti-mouse (A28175, Invitrogen) or AlexaFluor 594 donkey anti-rabbit (A32754, Invitrogen) secondary antibodies at RT followed by 5 min incubation with 1 μg/ml DAPI and 2 washes in PBS. For NADH autofluorescence studies, mice were gavaged with vehicle or 120 mg/kg sphingolipid-like compound 893 at ZT8.5 (4-10.5 h before sacrifice between ZT12.5 and ZT18). Post sacrifice, livers were excised, washed 3× with PBS, placed in DMEM supplemented with 10% FBS and 1% penicillin-streptomycin, and immediately imaged. NADH/NADPH autofluorescence was detected with 740 nm excitation and 450±50 nm detectors using a Mai Tai two-photon laser. Fluorescence microscopy was performed on a Zeiss LSM 780 confocal using a 63× oil objective with a 1.7 numerical aperture (NA) or using a Nikon TE2000-S inverted epifluorescence microscope with a 100× oil objective (1.3 NA) and a Photometrics CoolSNAP ES2 monochrome CCD camera. All confocal images are 16-bit images from 8-15 Z-stacks with 0.5 micron steps. At least 8-12 non-overlapping fields of view were obtained. Confocal images were obtained using Zeiss Zen 2.3 image acquisition software. To analyze the co-localization between DRP1/citrate synthase signals, Mander's overlap coefficient (MOC) was calculated using the JACOP co-localization plug-in of ImageJ v.1.52e (NIH) post background subtraction per field basis, 40 cells were analyzed from 2 biological replicates. For H&E staining, livers were fixed in formalin, dehydrated in ethanol, and processed by the Experimental Tissue Research pathology core facility at UCI and evaluated on a Nikon Ti2-F inverted epifluorescence microscope equipped with a DS-Fi3 color camera. Five non-overlapping fields were acquired from 3 different liver sections obtained from 3 mice per group (SD, HFD, or HFD+120 mg/kg sphingolipid-like compound 893). For imaging of brain mitochondria, mice were perfused transcardially with PBS followed by 4% paraformaldehyde immediately after euthanasia. Whole brains were removed, incubated in 4% paraformaldehyde at 4° C. for 24 h, and then transferred to a 30% sucrose solution in 0.1 M PBS for storage. To evaluate the arcuate nucleus (ARC) of the hypothalamus, the coordinates −0.5 to −2.4 mm were determined using a mouse brain atlas (Franklin, K. B. J. and Paxinos, G. (2001) The Mouse Brain in Stereotaxic Coordinates. 3^{rd} Edition, Academic Press, New York). A coronal slice was frozen in OCT on dry ice and 30 micron sections prepared, rehydrated with PBS, blocked and permeabilized with 5% normal goat serum in 0.3% Triton X-100 at 37° C. for 30 min, incubated for 24 h at 4° C. with citrate synthase primary antibody (1:100), washed, incubated with Alexa Fluor 488-conjugated secondary antibody (1:200), and counterstained with DAPI before mounting in Vectashield. No fluorescence was observed when secondary antibodies were omitted. Images from 5-10 non-overlapping fields in 2 different sections were evaluated from each of 4 mice per group (HFD+vehicle or HFD+120 mg/kg sphingolipid-like compound 893) using a Zeiss LSM 780 confocal microscope and a 63× oil objective.

### Morphometric Quantification of Mitochondrial Networks

Schematics describing the quantitative analysis of mitochondrial networks are provided in FIGS. 1 (in vitro) and 11 (in vivo). Analysis was performed using ImageJ software as described in (A. Chaudhry, R. Shi, & D. S. Luciani, Am J Physiol Endocrinol Metab. 318:E87-E101, 2020, the disclosure of which is incorporated herein by reference). Briefly, maximum projections from Z-stacks were pre-processed to remove background, manually thresholded as necessary to accurately capture mitochondria, and binarized images evaluated using the analyze particles tool (roundness=4×area/π×width and aspect ratio=width/height) or skeletonized and analyzed using the analyze skeleton 2D/3D function (branch length). Cell boundaries were manually delimited using the brightfield channel. For in vivo samples, noise was reduced with the despeckle function; branch length was not calculated for in vivo samples as hepatic mitochondria are minimally branched. In vitro analysis was performed on 40 cells (20 cells from each of the 2 biological replicates) from 6-10 non-overlapping fields of view. In each cell, 100-500 objects were evaluated and averaged; average values from 40 individual cells were used to generate averages for each condition. Analysis of liver and brain mitochondria was performed on a per field basis using 6-12 non-overlapping fields collected for each animal.

### Statistical Analysis

Mean±SEM is presented except where otherwise indicated in the legends. All experimental data is from >3 independent biological replicates except where otherwise indicated in the legends. Statistical analysis was performed using Graphpad Prism software except for lipid profiling when the statistical package R was used. Corrections for multiple comparisons were made as indicated in the legends and adjusted P-values reported: ns, not significant, P>0.05; *, P<0.05; **, P<0.01; ***, P<0.001.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. A method of treating a disorder or condition, comprising administering a sphingolipid-like compound to a subject having the disorder or condition, wherein the disorder or condition is related to metabolism.

2. The method as in claim 1, wherein the sphingolipid-like compound is based on O-benzyl pyrrolidines having the formula: 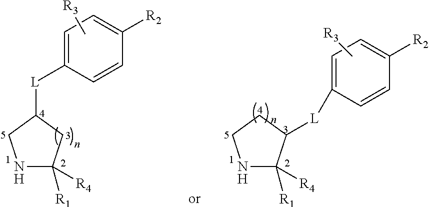
R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof;
R₂ is an aliphatic chain (C₆-C₁₀);
R₃ is a mono-, di-, tri- or quad-aromatic substituent comprising H, halogen, alkyl, alkoxy, azide (N₃), ether, NO₂, or cyanide (CN);
One of R₁ or R₄ is an alcohol (CH₂OH) or H;
L is O—CH₂; and
n is an independently selected integer selected from 1, 2, or 3.

3. The method as in claim 1, wherein the sphingolipid-like compound is based on diastereomeric 3- and 4-C-aryl pyrrolidines having the formula: 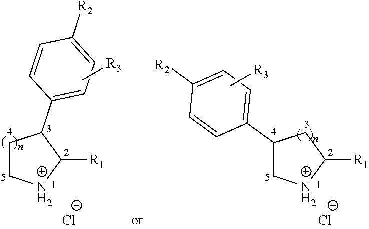
R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof;
R₂ is an aliphatic chain (C₆-C₁₄);
R₃ is a mono-, di-, tri- or tetra-aromatic substituent comprising hydrogen, halogen, alkyl, alkoxy, azide (N₃), ether, NO₂, or cyanide (CN); and
n is an independently selected integer selected from 1, 2, or 3.

4. The method as in claim 3, wherein the sphingolipid-like compound is compound 893 having the formula:

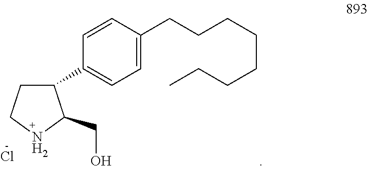 [SMILES: Cl ; CCCCCCCCc1ccc([C@H]2CCN[C@@H]2CO)cc1]

5. The method as in claim 3, wherein the sphingolipid-like compound is compound 1090 having the formula:

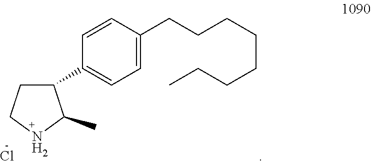 [SMILES: CCCCCCCCc1ccc([C@H]2CCN[C@@H]2C)cc1 ; Cl]

6. The method as in claim 1, wherein the sphingolipid-like compound is based on azacycles with an attached heteroaromatic appendage having the formula: 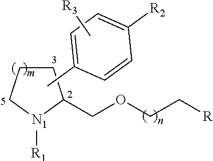
or a pharmaceutically acceptable salt thereof;
R is an optionally substituted heteroaromatic moiety such as an optionally substituted pyridazine, optionally substituted pyridine, optionally substituted pyrimidine, phenoxazine, or optionally substituted phenothiazine;
R₁ is H, alkyl such as C₁₋₆ alkyl or C₁₋₄ alkyl including methyl, ethyl, propyl, isopropyl, n-butyl, s-butyl, isobutyl, t-butyl, etc, Ac, Boc, guanidine moiety;
R₂ is an aliphatic chain comprising 6 to 14 carbons;
R₃ is a 1, 2, 3, or 4 substituents, wherein each substituent, independently, is H, halogen, alkyl, alkoxy, N₃, NO₂, and CN;
n is independently 1, 2, 3, or 4;
m is independently 1 or 2;
the phenyl moiety can be attached at any available position of the azacycle core; and
R is a 1,2-pyridazine having the formula: 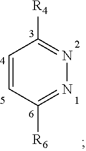
R₄ and R₅ are functional groups independently selected from: alkyl including methyl, optionally substituted aryl (i.e., unsubstituted aryl or substituted aryl) including optionally substituted phenyl, and optionally substituted heteroaryl including optionally substituted pyridine and optionally substituted pyrimidine; and
the pyridazine moiety is connected to the azacycle at the position 4 or 5 of the pyridazine.

7. The method of claim 6, wherein the sphingolipid-like compound is compound 325 having the formula:

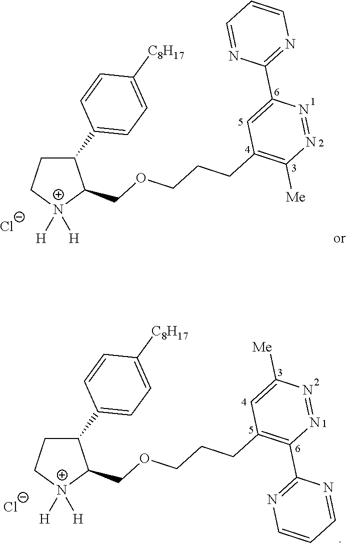

8. The method as in claim 1, wherein the sphingolipid-like compound is based on diastereomeric 2-C-aryl pyrrolidines having the formula: 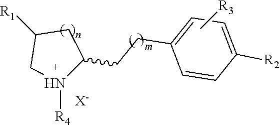
R₁ is a functional group selected from H, an alkyl chain, OH, (CH₂)ₙOH, CHOH-alkyl, CHOH-alkyne, (CH₂)ₙOR′, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof, where R′ is an alkyl, alkene or alkyne;
R₂ is an aliphatic chain (C₆-C₁₄);
R₃ is a mono-, di-, tri- or tetra-aromatic substituent that includes hydrogen, halogen, alkyl, alkoxy, azide (N₃), ether, NO₂, cyanide (CN), or a combination thereof;
R₄ is a functional group selected from H, alkyl including methyl (Me), ester, or acyl;
X⁻ is an anion of the suitable acid;
n is an independently selected integer selected from 1, 2, or 3; and
m is an independently selected integer selected from 0, 1 or 2.

9. The method as in any previous claim, wherein the disorder or condition comprises obesity.

10. The method as in any previous claim, wherein the disease or condition comprises metabolic syndrome.

11. The method as in any previous claim, wherein the disease or condition comprises hyperglycemia.

12. The method as in any previous claim, wherein the disease or condition comprises type 2 diabetes.

13. The method as in any previous claim, wherein the disease or condition comprises insulin resistance.

14. The method as in any previous claim, wherein the disease or condition comprises leptin resistance.

15. The method as in any previous claim, wherein the disease or condition comprises hyperleptinemia.

16. The method as in any previous claim, wherein the disease or condition comprises hepatic steatosis.

17. The method as in any previous claim, wherein the disease or condition comprises nonalcoholic steatohepatitis.

18. The method as in any previous claim, wherein the administering of the sphingolipid-like compound reduces the subject's food intake.

19. The method as in any previous claim, wherein the administering of the sphingolipid-like compound decreases weight gain in the subject.

20. The method as in any previous claim, wherein the administering of the sphingolipid-like compound decreases adiposity in the subject.

21. The method as in any previous claim, wherein the administering of the sphingolipid-like compound decreases metabolic dysfunction in the subject.

22. The method as in any previous claim, wherein the administering of the sphingolipid-like compound promotes insulin sensitivity in the subject.

23. The method as in any previous claim, wherein the administering of the sphingolipid-like compound promotes leptin sensitivity in the subject.

24. The method as in any previous claim, wherein the administering of the sphingolipid-like compound improves glucose tolerance.

25. The method as in any previous claim, wherein the administering of the sphingolipid-like compound reduces plasma leptin levels.

26. The method as in any previous claim, wherein the administering of the sphingolipid-like compound reduces plasma insulin levels.

27. The method as in any previous claim, wherein the administering of the sphingolipid-like compound reduces ceramide levels.

28. The method as in any previous claim, wherein the administering of the sphingolipid-like compound increases adiponectin levels.

29. The method as in any previous claim, wherein the administering of the sphingolipid-like compound reduces body fat.

30. The method as in any previous claim, wherein the administering of the sphingolipid-like compound resolves hepatic steatosis in the subject.

31. The method as in any previous claim, wherein the administering of the sphingolipid-like compound resolves steatohepatitis.

32. The method as in any previous claim, wherein the treatment is combined with an FDA-approved or EMA-approved standard of care.

33. The method as in any previous claim further comprising diagnosing the individual as having the condition or disorder.

34. A method of mitigating mitochondrial fragmentation, comprising:
contacting a biological cell with a sphingolipid-like compound, wherein the biological cell is undergoing mitochondrial fragmentation.

35. The method as in claim 34, wherein the sphingolipid-like compound is based on O-benzyl pyrrolidines having the formula: 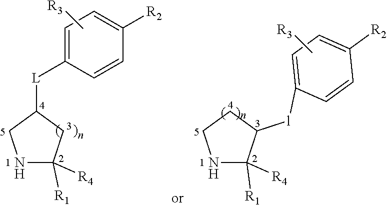
R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof;
R₂ is an aliphatic chain (C₆-C₁₀);
R₃ is a mono-, di-, tri- or quad-aromatic substituent comprising H, halogen, alkyl, alkoxy, azide (N₃), ether, NO₂, or cyanide (CN);
One of R₁ R₄ is an alcohol (CH₂OH) or H;
L is O—CH₂; and
n is an independently selected integer selected from 1, 2, or 3.

36. The method as in claim 34, wherein the sphingolipid-like compound is based on diastereomeric 3- and 4-C-aryl pyrrolidines having the formula: 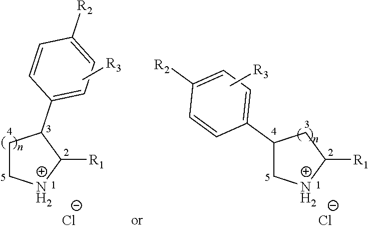
R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof;
R₂ is an aliphatic chain (C₆-C₁₄);
R₃ is a mono-, di-, tri- or tetra-aromatic substituent comprising hydrogen, halogen, alkyl, alkoxy, azide (N₃), ether, NO₂, or cyanide (CN); and
n is an independently selected integer selected from 1, 2, or 3.

37. The method as in claim 36, wherein the sphingolipid-like compound is compound 893 having the formula:

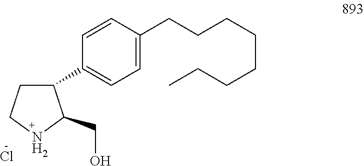 [SMILES: Cl ; CCCCCCCCc1ccc([C@H]2CCN[C@@H]2CO)cc1]

38. The method as in claim 36, wherein the sphingolipid-like compound is compound 1090 having the formula:

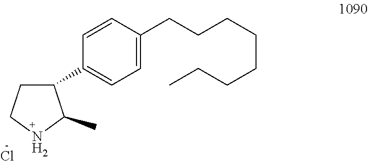 [SMILES: CCCCCCCCc1ccc([C@H]2CCN[C@@H]2C)cc1 ; Cl]

39. The method as in claim 34, wherein the sphingolipid-like compound is based on azacycles with an attached heteroaromatic appendage having the formula: 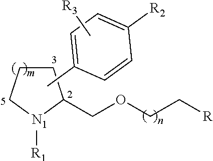
or a pharmaceutically acceptable salt thereof;
R is an optionally substituted heteroaromatic moiety such as an optionally substituted pyridazine, optionally substituted pyridine, optionally substituted pyrimidine, phenoxazine, or optionally substituted phenothiazine;
R₁ is H, alkyl such as C₁₋₆ alkyl or C₁₋₄ alkyl including methyl, ethyl, propyl, isopropyl, n-butyl, s-butyl, isobutyl, t-butyl, etc, Ac, Boc, guanidine moiety;
R₂ is an aliphatic chain comprising 6 to 14 carbons;
R₃ is a 1, 2, 3, or 4 substituents, wherein each substituent, independently, is H, halogen, alkyl, alkoxy, N₃, NO₂, and CN;
n is independently 1, 2, 3, or 4;
m is independently 1 or 2;
the phenyl moiety can be attached at any available position of the azacycle core; and
R is a 1,2-pyridazine having the formula: 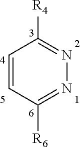
R₄ and R₅ are functional groups independently selected from: alkyl including methyl, optionally substituted aryl (i.e., unsubstituted aryl or substituted aryl) including optionally substituted phenyl, and optionally substituted heteroaryl including optionally substituted pyridine and optionally substituted pyrimidine; and
the pyridazine moiety is connected to the azacycle at the position 4 or 5 of the pyridazine.

40. The method of claim 39, wherein the sphingolipid-like compound is compound 325 having the formula:

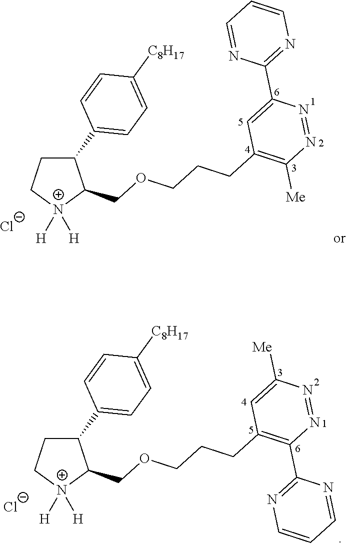

41. The method as in claim 34, wherein the sphingolipid-like compound is based on diastereomeric 2-C-aryl pyrrolidines having the formula: 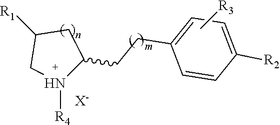
R₁ is a functional group selected from H, an alkyl chain, OH, (CH₂)ₙOH, CHOH-alkyl, CHOH-alkyne, (CH₂)ₙOR′, (CH₂)ₙPO(OH)₂ and esters thereof, CH═CHPO(OH)₂ and esters thereof, (CH₂CH₂)ₙPO(OH)₂ and esters thereof, and (CH₂)ₙOPO(OH)₂ and esters thereof, (CH₂)ₙPO₃ and esters thereof, where R′ is an alkyl, alkene or alkyne;
R₂ is an aliphatic chain (C₆-C₁₄);
R₃ is a mono-, di-, tri- or tetra-aromatic substituent that includes hydrogen, halogen, alkyl, alkoxy, azide (N₃), ether, NO₂, cyanide (CN), or a combination thereof;
R₄ is a functional group selected from H, alkyl including methyl (Me), ester, or acyl;
X⁻ is an anion of the suitable acid;
n is an independently selected integer selected from 1, 2, or 3; and
m is an independently selected integer selected from 0, 1 or 2.

42. The method as in claim 34, wherein the biological cell is associated a metabolic disorder or condition.

43. The method as in claim 42, wherein the disorder or condition comprises obesity.

44. The method as in claim 42 or 43, wherein the disease or condition comprises metabolic syndrome.

45. The method as in claim 42, 43, or 44, wherein the disease or condition comprises hyperglycemia.

46. The method as in any one of claims 42-45, wherein the disease or condition comprises type 2 diabetes.

47. The method as in any one of claims 42-46, wherein the disease or condition comprises insulin resistance.

48. The method as in any one of claims 42-47, wherein the disease or condition comprises leptin resistance.

49. The method as in any one of claims 42-48, wherein the disease or condition comprises hyperleptinemia.

50. The method as in any one of claims 42-49, wherein the disease or condition comprises hepatic steatosis.

51. The method as in any one of claims 42-50, wherein the disease or condition comprises nonalcoholic steatohepatitis.

52. The method as in any one of claims 34-51, wherein the contacting the biological cell with the sphingolipid-like compound reverses mitochondrial fragmentation.

53. A method of mitigating mitochondrial fragmentation, comprising:
contacting a biological cell with an ARF6 antagonist or a PIKfyve antagonist, wherein the biological cell is undergoing mitochondrial fragmentation.

54. The method of claim 52, wherein the ARF6 antagonist is NAV2729, SecinH3, perphenazine, or a derivative thereof.

55. The method of claim 52, wherein the PIKfyve antagonist is YM201636, APY0201, Apilimod, Late Endosome Trafficking Inhibitor EGA, or a derivative thereof.

56. The method as in claim 53, 54, or 55, wherein the contacting the biological cell with the ARF6 antagonist or the PIKfyve antagonist reverses mitochondrial fragmentation.

57. A method of treating a disorder or condition, comprising administering an ARF6 antagonist or a PIKfyve antagonist to a subject having the disorder or condition, wherein the disorder or condition is related to metabolism.

58. The method of claim 57, wherein the ARF6 antagonist is NAV2729, SecinH3, perphenazine, or a derivative thereof.

59. The method of claim 57, wherein the PIKfyve antagonist is YM201636, APY0201, Apilimod, Late Endosome Trafficking Inhibitor EGA, or a derivative thereof.
